(19) United States
(12) Patent Application Publication
(10) Pub. No.: US 2014/0106406 A1
(43) Pub. Date: Apr. 17, 2014

(54) GENE CLUSTER FOR THE PRODUCTION OF GOUGEROTIN, GOUGEROTIN ANALOGUES, AND PRECURSORS THEREOF

(71) Applicant: Bayer Cropscience LP, Davis, CA (US)

(72) Inventor: Daniel M. JOO, Davis, CA (US)

(73) Assignee: Bayer Cropscience LP, Davis, CA (US)

(21) Appl. No.: 14/054,503

(22) Filed: Oct. 15, 2013

Related U.S. Application Data
(60) Provisional application No. 61/714,267, filed on Oct. 16, 2012.

(51) Int. Cl. C12P 19/28

(52) U.S. Cl. CPC C12P 19/28
USPC 435/85; 536/23.1; 435/252.35; 435/320.1

(57) ABSTRACT

The present disclosure relates to the molecular cloning of a gougerotin biosynthetic gene cluster from Streptomyces microflavus, and identification of individual genes in the gene cluster as well as the proteins encoded thereby. A gougerotin gene cluster comprising 13 open reading frames (ORFs) is located within a genetic locus of Streptomyces microflavus. The gougerotin gene cluster further comprises another 11 ORFs, which are also disclosed. Gougerotin analogs and methods for producing them by manipulation of the gougerotin gene cluster and the genes therein are also disclosed.

### CROSS REFERENCE TO RELATED APPLICATION

This application claims the benefit of U.S. Provisional Application No. 61/714,267, filed Oct. 16, 2012, the contents of which are incorporated herein by reference in its entirety.

### FIELD OF INVENTION

The present invention relates to the field of bacterial strains and their ability to control plant diseases and pests.

### BACKGROUND OF INVENTION

Phytophagous mites, especially spider mites, are a major agricultural pest of orchards, greenhouses and many vegetable and fruit crops, including peppers, tomatoes, potatoes, squash, eggplant, cucumber and strawberries. Mites damage leaf and/or fruit surfaces using their sharp mouthparts. Besides direct damage to plant parts (referred to as stippling), mite feeding also causes increased susceptibility to plant diseases.

Mites are acari rather than insects, and few broad spectrum insecticides are also effective against mites. Characteristics of mites and of available miticides pose challenges to mite control. For example, spider mites, one of the most economically important families of mites, generally live on the undersides of leaves of plants, such that they are difficult to treat. Further, mites are known to develop resistance to presently available miticides, many of which have a single mode of action, within two to four years. Few available miticides have activity against mite eggs, making repeat applications necessary. Therefore, there is a need for new miticides having translaminar, ovicidal and strong residual activities in addition to good knockdown activity.

### SUMMARY OF INVENTION

The present invention provides the Streptomyces microflavus strain NRRL B-50550 or a phytophagous-miticidal mutant (strain) derived therefrom.

The present invention also provides a composition containing Streptomyces microflavus strain NRRL B-50550 or a phytophagous-miticidal mutant (strain) derived therefrom. In one aspect, the composition is a fermentation product of the Streptomyces microflavus strain NRRL B-50550 or a phytophagous-miticidal mutant strain derived therefrom.

The present invention also provides the gougerotin biosynthetic gene cluster from Streptomyces microflavus, the characterization of the individual genes in the gene cluster, and the proteins encoded thereby. A gougerotin gene cluster is disclosed, the gene cluster comprising 14 open reading frames (ORFs) referred to as ORFs 4251 to 4253, 4255 to 4259, 4261 to 4265, and 4271, respectively (SEQ ID NOS:1, 3, 5, 9, 11, 13, 15, 17, 21, 23, 25, 27, 29, and 41 respectively), and referred to herein as GouA, GouB, GouC, GouD, GouE, GouF, GouG, GouH, GouI, GouJ, GouK, GouL, GouM, and Gou N, respectively. The corresponding proteins are provided at SEQ ID NOS:2, 4, 6, 10, 12, 14, 16, 18, 22, 24, 26, 28, 30, and 42, respectively. The genomic DNA sequence comprising the gougerotin biosynthetic gene cluster and some of the flanking regions is provided in SEQ ID NO:43, and describes the locations of genes GouA through GouN.

The present invention also provides a method of treating a plant to control a plant disease or pest, wherein the method comprises applying the Streptomyces microflavus strain NRRL-50550 or a phytophagous-miticidal mutant strain derived therefrom, to the plant, to a part of the plant and/or to a locus of the plant. In one embodiment, a fermentation product of the strain or a fermentation product of a mutant derived therefrom is applied to the plant and/or to a locus of the plant.

The invention also provides for a method of controlling phytophagous acari or insects comprising applying to a plant or to soil surrounding the plant the Streptomyces microflavus strain NRRL B-50550 or a phytophagous-miticidal strain derived therefrom. In one embodiment, a fermentation product of the strain or a fermentation product of a mutant derived thereform is applied to the plant and/or to a locus of the plant.

### BRIEF DESCRIPTION OF THE DRAWINGS

FIG. 1 shows the chemical structure of gougerotin, as well as the serine, sugar, cytosine, and sarcosine subdomains thereof.

FIG. 2 shows a potential biosynthetic pathway for gougerotin.

FIG. 3 shows the chemical structure of gougerotin, annotated with the open reading frame (ORF) numbers potentially involved with and/or responsible for particular subdomain structure of gougerotin.

### SEQUENCE LISTINGS

SEQ ID NO:1 is the nucleotide sequence of ORF 4251 (GouA), corresponding to the reverse complement of nucleotides 4455 to 4880 of SEQ ID NO:43.

SEQ ID NO:2 is the amino acid sequence encoded by the nucleotide sequence of ORF 4251 (SEQ ID NO:1).

SEQ ID NO:3 is the nucleotide sequence of ORF 4252 (GouB), corresponding to nucleotides 6340 to 6801 of SEQ ID NO:43.

SEQ ID NO:4 is the amino acid sequence encoded by the nucleotide sequence of ORF 4252 (SEQ ID NO:3).

SEQ ID NO:5 is the nucleotide sequence of ORF 4253 (GouC), corresponding to nucleotides 7197 to 7712 of SEQ ID NO:43.

SEQ ID NO:6 is the amino acid sequence encoded by the nucleotide sequence of ORF 4253 (SEQ ID NO:5).

SEQ ID NO:7 is the nucleotide sequence of ORF 4254, corresponding to the reverse complement of nucleotides 8130 to 8486 of SEQ ID NO:43.

SEQ ID NO:8 is the amino acid sequence encoded by the nucleotide sequence of ORF 4254 (SEQ ID NO:7).

SEQ ID NO:9 is the nucleotide sequence of ORF 4255 (GouD), corresponding to nucleotides 8589 to 8729 of SEQ ID NO:43.

SEQ ID NO:10 is the amino acid sequence encoded by the nucleotide sequence of ORF 4255 (SEQ ID NO:9).

SEQ ID NO:11 is the nucleotide sequence of ORF 4256 (GouE), corresponding to nucleotides 8912 to 9826 of SEQ ID NO:43.

SEQ ID NO:12 is the amino acid sequence encoded by the nucleotide sequence of ORF 4256 (SEQ ID NO:11).

SEQ ID NO:13 is the nucleotide sequence of ORF 4257 (GouF), corresponding to nucleotides 9827 to 10823 of SEQ ID NO:43.

SEQ ID NO:14 is the amino acid sequence encoded by the nucleotide sequence of ORF 4257 (SEQ ID NO:13).

SEQ ID NO:15 is the nucleotide sequence of ORF 4258 (GouG), corresponding to nucleotides 10820 to 12013 of SEQ ID NO:43.

SEQ ID NO:16 is the amino acid sequence encoded by the nucleotide sequence of ORF 4258 (SEQ ID NO:15).

SEQ ID NO:17 is the nucleotide sequence of ORF 4259 (GouH), corresponding to nucleotides 12020 to 13145 of SEQ ID NO:43.

SEQ ID NO:18 is the amino acid sequence encoded by the nucleotide sequence of ORF 4259 (SEQ ID NO:17).

SEQ ID NO:19 is the nucleotide sequence of ORF 4260, corresponding to the reverse complement of nucleotides 13146 to 13265 of SEQ ID NO:43.

SEQ ID NO:20 is the amino acid sequence encoded by the nucleotide sequence of ORF 4260 (SEQ ID NO:19).

SEQ ID NO:21 is the nucleotide sequence of ORF 4261 (GouI), corresponding to nucleotides 13219 to 14334 of SEQ ID NO:43.

SEQ ID NO:22 is the amino acid sequence encoded by the nucleotide sequence of ORF 4261 (SEQ ID NO:21).

SEQ ID NO:23 is the nucleotide sequence of ORF 4262 (GouJ), corresponding to nucleotides 14350 to 15063 of SEQ ID NO:43.

SEQ ID NO:24 is the amino acid sequence encoded by the nucleotide sequence of ORF 4262 (SEQ ID NO:23).

SEQ ID NO:25 is the nucleotide sequence of ORF 4263 (GouK), corresponding to nucleotides 15411 to 16046 of SEQ ID NO:43.

SEQ ID NO:26 is the amino acid sequence encoded by the nucleotide sequence of ORF 4263 (SEQ ID NO:25).

SEQ ID NO:27 is the nucleotide sequence of ORF 4264 (GouL), corresponding to nucleotides 16142 to 17482 of SEQ ID NO:43.

SEQ ID NO:28 is the amino acid sequence encoded by the nucleotide sequence of ORF 4264 (SEQ ID NO:27).

SEQ ID NO:29 is the nucleotide sequence of ORF 4265 (GouM), corresponding to nucleotides 17549 to 19312 of SEQ ID NO:43.

SEQ ID NO:30 is the amino acid sequence encoded by the nucleotide sequence of ORF 4265 (SEQ ID NO:29).

SEQ ID NO:31 is the nucleotide sequence of ORF 4266, corresponding to nucleotides 19461 to 19574 of SEQ ID NO:43.

SEQ ID NO:32 is the amino acid sequence encoded by the nucleotide sequence of ORF 4266 (SEQ ID NO:31).

SEQ ID NO:33 is the nucleotide sequence of ORF 4267, corresponding to nucleotides 20147 to 20551 of SEQ ID NO:43.

SEQ ID NO:34 is the amino acid sequence encoded by the nucleotide sequence of ORF 4267 (SEQ ID NO:33).

SEQ ID NO:35 is the nucleotide sequence of ORF 4268.

SEQ ID NO:36 is the amino acid sequence encoded by the nucleotide sequence of

ORF 4268 (SEQ ID NO:35).

SEQ ID NO:37 is the nucleotide sequence of ORF 4269.

SEQ ID NO:38 is the amino acid sequence encoded by the nucleotide sequence of ORF 4269 (SEQ ID NO:37).

SEQ ID NO:39 is the nucleotide sequence of ORF 4270.

SEQ ID NO:40 is the amino acid sequence encoded by the nucleotide sequence of ORF 4270 (SEQ ID NO:39).

SEQ ID NO:41 is the nucleotide sequence of ORF 4271.

SEQ ID NO:42 is the amino acid sequence encoded by the nucleotide sequence of ORF 4271 (SEQ ID NO:41).

SEQ ID NO:43 is the nucleic acid sequence of a genetic locus comprising a gougerotin gene cluster.

### DETAILED DESCRIPTION OF INVENTION

All publications, patents and patent applications, including any drawings and appendices, herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

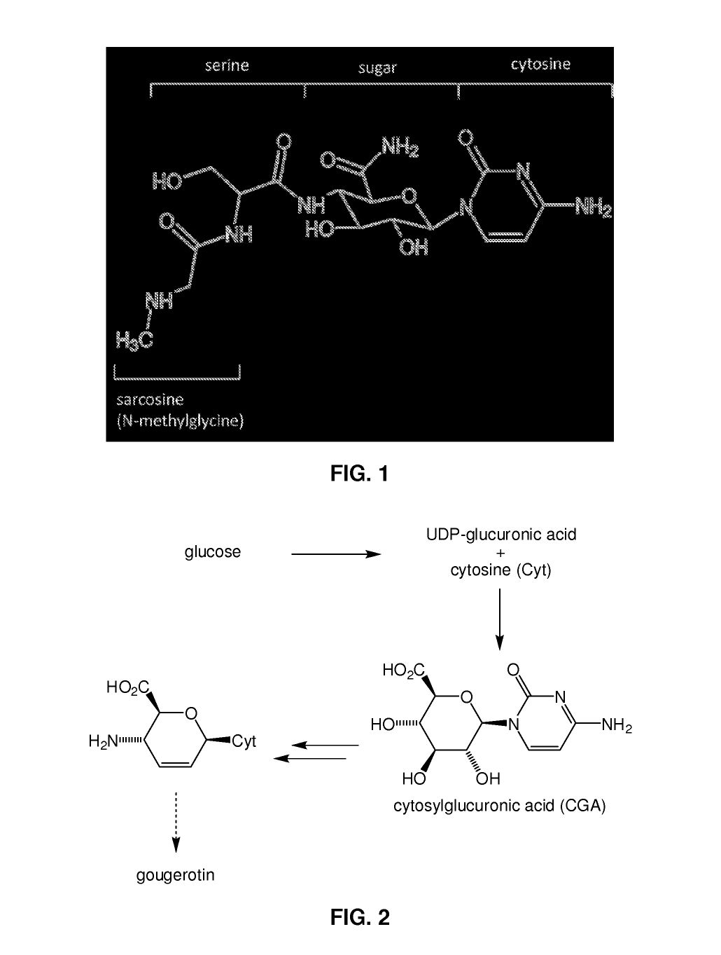

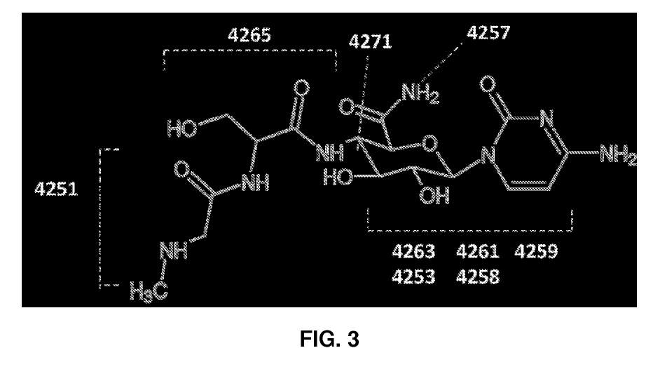

The present invention provides the Streptomyces microflavus strain NRRL B-50550 or a phytophagous-miticidal mutant strain derived therefrom. It has been found that the strain NRRL B-50550 has a variety of advantageous properties. Not only does the strain NRRL B-50550 (or its fermentation product) have acaricidal activity as such but, for example, also shows a high UV stability, a good translaminar activity, good ovicidal activity, long residual activity, drench activity as well as activity against a broad range of mites (see Example Section) and thus meets the requirements for an effective acaricide. In addition, the strain NRRL B-50550 (or its fermentation product) possesses both insecticidal activity and activity against various fungal phytopathogens such as leaf rust and mildew. This unique combination of activities makes the strain NRRL B-50550 a highly versatile candidate and renders the strain suitable to be broadly employed in methods of treating plants to control a plant disease and/or a plant pest. Such a broad range of activities and possible applications in agriculture has not yet been reported for known Streptomyces strains. In relation to a possible agricultural use, streptomyces strains have been predominantly described in publications from the late 1960's and early 1970s. See, for example, the British Patent No. GB 1 507 193 that describes the Streptomyces rimofaciens strain No. B-98891, deposited as ATCC 31120, which produces the antibiotic B-98891. According to GB 1 507 193, filed March 1975, the antibiotic B-98891 is the active ingredient that provides antifungal activity of the Streptomyces rimofaciens strain No. B-98891 against powdery mildew. U.S. Pat. No. 3,849,398, filed Aug. 2, 1972, describes that the strain Streptomyces toyocaensis var. aspiculamyceticus produces the antibiotic aspiculamycin which is also known as gougerotin (see, Toru Ikeuchi et al., 25 J. ANTIBIOTICS 548 (September 1972). According to U.S. Pat. No. 3,849,398, gougerotin has parasiticidal action against parasites on animals, such as pin worm and the like, although gougerotin is said to show a weak antibacterial activity against gram-positive, gram-negative bacteria and tubercule bacillus. Similarly, Japanese Patent Application No. JP 53109998 (A), published 1978, reports the strain Streptomyces toyocaensis (LA-681) and its ability to produce gougerotin for use as miticide. However, it is to be noted that no miticidal product based on such Streptomyces strains is commercially available. Thus, the Streptomyces microflavus strain NRRL B-50550 with its broad efficacy against acari (based on gougerotin production), fungi and insects and its favorable properties in terms of mode of action (e.g., translaminar activity and residual activity) represents a significant and unexpected advancement in terms of biological and advantageous properties which as such have not been reported for known Streptomyces strains. Additionally, Applicant has discovered the Streptomyces microflavus gene cluster responsible for gougerotin production. The structure of gougerotin is shown below, and at FIG. 1.

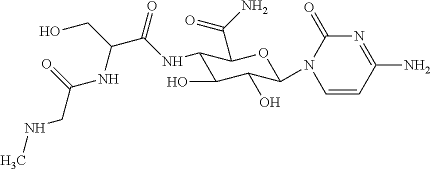 [SMILES: CNCC(=O)NC(CO)C(=O)NC1C(C(N)=O)OC(n2ccc(N)nc2=O)C(O)C1O]

The microorganisms and particular strains described herein, unless specifically noted otherwise, are all separated from nature (i.e., isolated) and grown under artificial conditions, such as in shake flask cultures or through scaled-up manufacturing processes, such as in bioreactors, as described herein.

In one embodiment, a phytophagous-miticidal mutant strain of the Streptomyces microflavus strain NRRL B-50550 is provided. The term “mutant” refers to a genetic variant derived from Streptomyces microflavus strain NRRL B-50550. In one embodiment, the mutant has one or more or all the identifying (functional) characteristics of Streptomyces microflavus strain NRRL B-50550. In a particular instance, the mutant or a fermentation product thereof controls (as an identifying functional characteristic) mites at least as well as the parent Streptomyces microflavus NRRL B-50550 strain. In addition, the mutant or a fermentation product thereof may have one, two, three, four or all five of the following characteristics: translaminar activity in relation to the miticidal activity, residual activity in relation to the miticidal activity, ovicidal activity, insecticide activity, in particular against diabrotica, or activity against fungal phytopathogens, in particular against mildew and rust disease. Such mutants may be genetic variants having a genomic sequence that has greater than about 85%, greater than about 90%, greater than about 95%, greater than about 98%, or greater than about 99% sequence identity to Streptomyces microflavus strain NRRL B-50550. Mutants may be obtained by treating Streptomyces microflavus strain NRRL B-50550 cells with chemicals or irradiation or by selecting spontaneous mutants from a population of NRRL B-50550 cells (such as phage resistant or antibiotic resistant mutants) or by other means well known to those practiced in the art.

Suitable chemicals for mutagenesis of Streptomyces microflavus include hydroxylamine hydrochloride, methyl methanesulfonate (MMS), ethyl methanesulfonate (EMS), 4-nitroquinoline 1-oxide (NQO), mitomycin C or N-methyl-N′-nitro-N-nitrosoguanidine (NTG), to mention only a few (cf., for example, Stonesifer & Baltz, Proc. Natl. Acad. Sci. USA Vol. 82, pp. 1180-1183, February 1985). The mutagenesis of Streptomyces strains by, for example, NTG, using spore solutions of the respective Streptomyces strain is well known to the person skilled in the art. See, for example Delic et al, Mutation Research/Fundamental and Molecular Mechanisms of Mutagenesis, Volume 9, Issue 2, February 1970, pages 167-182, or Chen et al., J Antibiot (Tokyo), 2001 November; 54(11), pages 967-972.). In more detail, Streptomyces microflavus can be subjected to mutation by NTG using the protocol described in Kieser, T., et al., 2000, supra. Practical Streptomyces Genetics, Ch. 5 John Innes Centre, Norwich Research Park, England (2000), pp. 99-107. Mutagenesis of spores of Streptomyces microflavus by ultraviolet light (UV) can be carried out using standard protocols. For example, a spore suspension of the Streptomyces strain (freshly prepared or frozen in 20% glycerol) can be suspended in a medium that does not absorb UV light at a wave length of 254 nm (for example, water or 20% glycerol are suitable). The spore suspension is then placed in a glass Petri dish and irradiated with a low pressure mercury vapour lamp that emits most of its energy at 254 nm with constant agitation for an appropriate time at 30° C. (the most appropriate time of irradiation can be determined by first plotting a dose-survival curve). Slants or plates of non-selective medium can, for example, then be inoculated with the dense irradiated spore suspension and the so obtained mutant strains can be assessed for their properties as explained in the following. See Kieser, T., et al. 2000, supra.

The mutant strain can be any mutant strain that has one or more or all the identifying characteristics of Streptomyces microflavus strain NRRL B-50550 and in particular miticidal activity that is comparable or better than that of Streptomyces microflavus NRRL B-50550. The miticidal activity can, for example, be determined against two-spotted spider mites (“TSSM”) as explained in Example 2 herein, meaning culture stocks of the mutant strain of Streptomyces microflavus NRRL B-50550 can be grown in 1 L shake flasks in Media 1 or Media 2 of Example 2 at 20-30° C. for 3-5 days, and the diluted fermentation product can then be applied on top and bottom of lima bean leaves of two plants, after which treatment, plants can be infested on the same day with 50-100 TSSM and left in the greenhouse for five days.

In one aspect, of the invention, the Streptomyces microflavus strain NRRL B-50550 or a phytophagous-miticidal mutant strain thereof has translaminar activity. The term “translaminar activity” is used herein in its regular meaning in the art and thus by “translaminar activity” is meant the ability of a compound or composition (here a composition such as a fermentation product containing the Streptomyces microflavus strain NRRL B-50550 or a mutant strain thereof) of moving through the leaf tissue of the plant to be treated. A translaminar compound/composition penetrates leaf tissues and forms a reservoir of active ingredient within the leaf. This translaminar activity therefore also provides residual activity against foliar-feeding insects and mites. Because the composition (or its one or more active ingredients) can move through leaves, thorough spray coverage is less critical to control acari such as mites, which normally feed on leaf undersides. The translaminar activity of a mutant strain alone or in comparison to Streptomyces microflavus NRRL B-50550 can, for example, be determined against two-spotted spider mites (“TSSM”) as explained in Example 6 herein.

In another aspect of the invention, the Streptomyces microflavus strain NRRL B-50550 or a phytophagous-miticidal mutant strain thereof has residual activity. The term “residual activity” is used herein in its regular meaning in the art and thus by “residual activity” is meant the ability of a compound or composition (here a composition such as a fermentation product containing the Streptomyces microflavus strain NRRL B-50550 or a mutant strain thereof) to remain effective for an extended period of time after it is applied. The length of time may depend on the formulation (dust, liquid, etc.), the type of plant or location and the condition of the plant surface or soil surface (wet, dry, etc.) to which a composition containing Streptomyces microflavus strain NRRL B-50550 or a mutant strain thereof is applied. The residual activity of a mutant strain alone or in comparison to Streptomyces microflavus NRRL B-50550 can, for example, be determined against two-spotted spider mites (“TSSM”) as explained in Example 2 or 7 herein and means, in relation to the miticidal effect, that an antimiticidal effect can still be observed after several days (e.g., 12 days) under the conditions of Example 2 or 5.

In another aspect of the invention, the Streptomyces microflavus strain NRRL B-50550 or a phytophagous-miticidal mutant strain thereof has ovicidal activity. The term “ovicidal activity” is used herein in its regular meaning in the art to mean “the ability of causing destruction or death of an ovum” and is used herein in relation to eggs of acari such as mites. The ovicidal activity of a mutant strain of Streptomyces microflavus NRRL B-50550 alone or in comparison to Streptomyces microflavus NRRL B-50550 can be determined using the method as described in Example 7.

In another aspect of the invention, the Streptomyces microflavus strain NRRL B-50550 or a phytophagous-miticidal mutant strain thereof may have drench activity. The term “drench activity” is used herein in its regular meaning in the art to mean pesticidal activity that travels from soil or other growth media upward through the plant via the xylem. The drench activity of a mutant strain of Streptomyces microflavus NRRL B-50550 alone or in comparison to Streptomyces microflavus NRRL B-5055 can be determined using the method as described in Example 8.

In another aspect of the invention, the Streptomyces microflavus strain NRRL B-50550 or a phytophagous-miticidal mutant strain thereof has miticidal activity against a variety of mite species, including, as illustrated in the Examples, but not limited to, activity against two-spotted spider mites, activity against citrus rust mites (Phyllocoptruta oleivora), eriophyid (russet) mites and broad mites.

The Streptomyces microflavus strain NRRL B-50550 or a phytophagous-miticidal mutant strain thereof may thus have activity against a mite that is selected from the group consisting of clover mite, brown mite, hazelnut spider mite, asparagus spider mite, brown wheat mite, legume mite, oxalis mite, boxwood mite, Texas citrus mite, Oriental red mite, citrus red mite, European red mite, yellow spider mite, fig spider mite, Lewis spider mite, six-spotted spider mite, Willamette mite, Yuma spider mite, web-spinning mite, pineapple mite, citrus green mite, honey-locust spider mite, tea red spider mite, southern red mite, avocado brown mite, spruce spider mite, avocado red mite, Banks grass mite, carmine spider mite, desert spider mite, vegetable spider mite, tumid spider mite, strawberry spider mite, two-spotted spider mite, McDaniel mite, Pacific spider mite, hawthorn spider mite, four-spotted spider mite, Schoenei spider mite, Chilean false spider mite, citrus flat mite, privet mite, flat scarlet mite, white-tailed mite, pineapple tarsonemid mite, West Indian sugar cane mite, bulb scale mite, cyclamen mite, broad mite, winter grain mite, red-legged earth mite, filbert big-bud mite, grape erineum mite, pear blister leaf mite, apple leaf edgeroller mite, peach mosaic vector mite, alder bead gall mite, Perian walnut leaf gall mite, pecan leaf edgeroll mite, fig bud mite, olive bud mite, citrus bud mite, litchi erineum mite, wheat curl mite, coconut flower and nut mite, sugar cane blister mite, buffalo grass mite, bermuda grass mite, carrot bud mite, sweet potato leaf gall mite, pomegranate leaf curl mite, ash sprangle gall mite, maple bladder gall mite, alder erineum mite, redberry mite, cotton blister mite, blueberry bud mite, pink tea rust mite, ribbed tea mite, grey citrus mite, sweet potato rust mite, horse chestnut rust mite, citrus rust mite, apple rust mite, grape rust mite, pear rust mite, flat needle sheath pine mite, wild rose bud and fruit mite, dryberry mite, mango rust mite, azalea rust mite, plum rust mite, peach silver mite, apple rust mite, tomato russet mite, pink citrus rust mite, cereal rust mite, rice rust mite and combinations thereof.

In another aspect of the invention, the Streptomyces microflavus , strain NRRL B-50550 or a phytophagous-miticidal mutant strain thereof may have also insecticide activity. The target insect may be a diabrotica. The diabrotica may be Banded cucumber beetle (Diabrotica balteata), Western spotted cucumber beetle (Diabrotica undecimpunctata undecimpunctata), or a corn rootworm such as Northern corn rootworm (Diabrotica barberi), Southern corn rootworm (Diabrotica undecimpunctata howardi), Western cucumber beetle (Diabrotica undecimpunctata tenella), Western corn rootworm (Diabrotica virgifera virgifera), Mexican corn rootworm (Diabrotica virgifera zeae) and combinations of such diabrotica.

In another aspect of the invention, the Streptomyces microflavus strain NRRL B-50550 or a phytophagous-miticidal mutant strain thereof has fungicide activity, meaning activity against a plant disease that is caused by a fungus. The plant disease may be mildew or a rust disease. Examples of mildew that can be treated with the Streptomyces microflavus strain NRRL B-50550 or a phytophagous-miticidal mutant strain thereof include, but are not limited to, powdery mildew, such as cucumber powdery mildew caused by Sphaerotbeca fuliginea, or downy mildew, such as brassica downy mildew, caused by Peronospora parasitica. Examples of a rust disease that may be treated with Streptomyces microflavus strain NRRL B-50550 or a phytophagous-miticidal mutant strain thereof include, but are not limited to, wheat leaf rust caused by Puccinia triticina (also known as P. recondita), wheat stem rust caused by Puccinia grammis, wheat stripe rust caused by Puccinia striiformis, leaf rust of barley caused by Puccinia hordei, leaf rust of rye caused by Puccinia recondita, brown leaf rust, crown rust, and stem rust.

Gougerotin Gene Cluster, ORFs, and Proteins Encoded thereby

The present disclosure provides the nucleic acid sequence of a gougerotin gene cluster located within a genetic locus, the ORFs contained therein, and the proteins encoded thereby. This information enables, for example, the isolation of related nucleic acid molecules encoding homologs of the gougerotin gene cluster and the corresponding ORFs, such as in other Streptomyces spp. This disclosure further enables the production of variants of the proteins (including, but not limited to GouA, GouB, GouC, GouD, GouE, GouF, GouG, GouH, GouI, GouJ, GouK, GouL, GouM, and/or GouN) encoded by a gougerotin gene cluster or portions thereof, and nucleic acid molecules encoding such variants.

The gougerotin gene cluster included within SEQ ID NO:43 (nucleotide residues 1-21933) includes twenty-four ORFs referred to as ORFs 4248 to 4271. ORFs 4251, 4252, 4253, 4255, 4256, 4257, 4258, 4259, 4261, 4262, 4263, 4264, 4265, and 4271 are thirteen genes gouA, gouB, gouC, gouD, gouE, gouF, gouG, gouH, gouI, gouJ, gouK, gouL, gouM, and gouN, respectively. The potential function of these genes and their possible role in gougerotin synthesis is provided in TABLE 1.

TABLE 1
Possible Role of Gougerotin Biosynthetic Genes

| | # of | | | |
|---|---|---|---|---|
| ORF | a.a. | Potential function | Strand | Possible role in gougerotin biosynthesis |
| 4248 | 58 | hypothetical protein | + | |
| 4249 | 568 | endo-1,4-beta- | − | |
| | | xylanase A precursor | | |
| 4250 | 99 | transposase | − | |
| 4251 | 141 | methyltransferase | − | may be involved in synthesis of sarcosine from glycine |
| 4252 | 153 | kinase | + | transfers a phosphate group |
| 4253 | 171 | dehydrogenase | + | may be involved in producing UDP-glucuronic acid |
| 4254 | 118 | hypothetical protein | − | |
| 4255 | 46 | hypothetical protein | + | |
| 4256 | 304 | hypothetical protein | + | |
| 4257 | 326 | aminotransferase | + | transfers an amino group to the sugar backbone or |
| | | | | involved in the synthesis of serine or sarcosine; has |
| | | | | similarity to DegT/DnrJ/EryC1/StrS aminotransferase |
| | | | | family which includes StsC, the aminotransferase |
| | | | | catalyzing the first amino transfer in the biosynthesis of |
| | | | | streptidine subunit of streptomycin |
| 4258 | 397 | similar to cytosinine | + | possible enzyme for creating cytosinine-like molecule; |
| | | synthase | | has similarity to DegT/DnrJ/EryC1/StrS |
| | | | | aminotransferase family |
| 4259 | 378 | phosphatase | + | may remove the phosphate group in UDP-glucuronic |
| | | | | acid to create a precursor to CGA |
| 4260 | 39 | hypothetical protein | − | |
| 4261 | 371 | CGA synthase | + | potential enzyme for synthesizing cytosylglucoronic |
| | | | | acid, a potential intermediate in gougerotin |
| 4262 | 237 | nucleotide binding | + | |
| 4263 | 211 | glycosyltransferase | + | Potentially another enzyme for attaching the sugar |
| | | | | group to cytosine to create CGA |
| 4264 | 446 | unknown | + | |
| 4265 | 587 | asparagine synthase | + | similar to asparagine synthase of other gram positive |
| | | | | bacteria in other genus; may synthesize one of the |
| | | | | amino acids in gougerotin |
| 4266 | 37 | hypothetical protein | + | |
| 4267 | 134 | hypothetical protein | + | |
| 4268 | 414 | transposase | + | |
| 4269 | 378 | transposase | − | |
| 4270 | 187 | transcription | − | |
| | | regulator | | |
| 4271 | 368 | monooxygenase | + | may transfer a hydroxyl group to the sugar backbone |

With the provision herein of the sequences of the disclosed gene locus (SEQ ID NO:43) and the ORFs contained therein, in vitro nucleic acid amplification (including, but not limited to, PCR) may be utilized as a simple method for producing nucleic acid sequences encoding one or more of the gougerotin biosynthetic proteins listed in TABLE 1. The following provides representative techniques for preparing a protein-encoding nucleic acid molecule in this manner.

RNA or DNA is extracted from cells by any one of a variety of methods well known to those of ordinary skill in the art. Sambrook et al. (in Molecular Cloning: A Laboratory Manual, Cold Spring Harbor Laboratory Press, New York, 1989) and Ausubel et al. (in Current Protocols in Molecular Biology, Greene Publ. Assoc. and Wiley-Intersciences, 1992) provide representative descriptions of methods for RNA or DNA isolation. The gougerotin biosynthetic enzymes are expressed, at least, in Streptomyces microflavus. Thus, in some examples, RNA or DNA may be extracted from Streptomyces microflavus cells. Extracted RNA may be used, for example, as a template for performing reverse transcription (RT)-PCR amplification to produce cDNA. Representative methods and conditions for RT-PCR are described by Kawasaki et al. (in PCR Protocols, A Guide to Methods and Applications, Innis et al. (eds.) 21-27 Academic Press, Inc., San Diego, Calif., 1990).

The selection of amplification primers may be made according to the portion(s) of the DNA to be amplified. In one embodiment, primers may be chosen to amplify a segment of DNA (e.g., a specific ORF or set of adjacent ORFs) or, in another embodiment, the entire DNA molecule. Variations in amplification conditions may be required to accommodate primers and amplicons of differing lengths and composition. Such considerations are well known in the art and are discussed for instance in Innis et al. (PCR Protocols, A Guide to Methods and Applications, Academic Press, Inc., San Diego, Calif., 1990). By way of example, the nucleic acid molecules encoding selected gougerotin biosynthetic proteins (such as any one or combination of, gouA through gouN) may be amplified using primers directed to the 5′- and 3′-ends of the prototypical Streptomyces microflavus gouA, gouB, gouC, gouD, gouE, gouF, gouG, gouH, gouI, gouJ, gouK, gouL, gouM, and/or you N sequences. It will be appreciated that many different primers may be derived from the provided nucleic acid sequences. Re-sequencing of amplification products obtained by any amplification procedure is recommended to facilitate confirmation of the amplified sequence and to provide information on natural variation between a gougerotin and amplified sequence. Oligonucleotides derived from any of the gougerotin sequences may be used in sequencing, for instance, the corresponding gougerotin (or gougerotin-related) amplicon.

In addition, both conventional hybridization and PCR amplification procedures may be employed to clone sequences encoding orthologs of the gougerotin gene cluster, or gougerotin ORFs (for example, one or more of SEQ ID NOs:1, 3, 5, 7, 9, 11, 13, 15, 17, 19, 21, 23, 25, 27, 29, 31, 33, 35, 37, 39, and 41). Common to both of these techniques is the hybridization of probes or primers that are derived from the gougerotin gene cluster with or without the upstream and downstream flanking regions or gougerotin ORF nucleic acid sequences. Furthermore, the hybridization may occur in the context of Northern blots, Southern blots, or PCR.

Direct PCR amplification may be performed on DNA libraries prepared from the bacterial species in question, or RT-PCR may be performed using RNA extracted from the bacterial cells using standard methods. PCR primers will comprise at least 10 consecutive nucleotides of the gougerotin gene cluster with or without the upstream and downstream flanking regions or gougerotin ORF nucleic acid sequences. One of skill in the art will appreciate that sequence differences between the gougerotin gene cluster or gougerotin ORF nucleic acid sequences and the target nucleic acid to be amplified may result in lower amplification efficiencies. To compensate for this, longer PCR primers or lower annealing temperatures may be used during the amplification cycle. Whenever lower annealing temperatures are used, sequential rounds of amplification using nested primer pairs may be useful to enhance amplification specificity.

Orthologs of the disclosed gougerotin biosynthetic proteins may be present in a number of other members of the Streptomyces genus, in other strains of the Streptomyces microflavus species, and in other gougerotin-producing organisms. With the provision of the nucleic acid sequence of the disclosed gougerotin gene cluster and its ORFs 4251-4253, 4255-4259, 4261-4265, and 4271, as well as flanking and intervening ORFs 4248-4250 and 4266-4270, the cloning by standard methods of protein-encoding DNA (such as, ORFs) and gene clusters that encode gougerotin biosynthetic enzyme orthologs in these other organisms is now enabled. Orthologs of the disclosed gougerotin biosynthetic enzymes and proteins have a biological activity or function as disclosed herein, including for example cytosine synthase (ORF 4258; gouG; SEQ ID NOs:15 & 16) or CGA synthase (ORF 4261; gouI; SEQ ID NOs:21 & 22).

Orthologs will generally share at least 65% sequence identity with the nucleic acid sequences encoding the disclosed gougerotin biosynthetic proteins (for example, one or more of SEQ ID NOs:1, 3, 5, 7, 9, 11, 13, 15, 17, 19, 21, 23, 25, 27, 29, 31, 33, 35, 37, 39, and 41). In specific embodiments, orthologous gougerotin gene clusters or gougerotin ORFs may share at least 70%, at least 75%, at least 80% at least 85%, at least 90%, at least 91%, at least 92%, at least 93%, at least 94%, at least 95%, at least 96%, at least 97%, at least 98%, or at least 99% sequence identity with the disclosed Streptomyces microflavus nucleotide or amino acid sequences, as applicable.

For conventional hybridization techniques the hybridization probe is preferably conjugated with a detectable label such as a radioactive label, and the probe is preferably at least 10 nucleotides in length. As is well known in the art, increasing the length of hybridization probes tends to provide enhanced specificity. A labeled probe derived from a gougerotin gene cluster or from gougerotin ORF nucleic acid sequences may be hybridized to a bacterial DNA library and the hybridization signal detected using methods known in the art. The hybridizing colony or plaque (depending on the type of library used) may be purified and the cloned sequence contained in that colony or plaque isolated and characterized.

In specific examples, genomic library construction can be accomplished rapidly using a variety of cosmid or fosmid systems that are commercially available (e.g., Stratagene, Epicentre). Advantageously, these systems minimize instability of the cloned DNA. In such examples, genomic library screening is followed by cosmid or fosmid isolation, grouping into families of overlapping clones and analysis to establish cluster identity. Cosmid end sequencing can be used to obtain preliminary information regarding the relevance of a particular clone based on expected pathway characteristics predicted from the natural product structure and its presumed biosynthetic origin.

Orthologs of a gougerotin gene cluster (+/−upstream or downstream flanking regions) or gougerotin ORF nucleic acid sequences alternatively may be obtained by immunoscreening of an expression library. With the provision herein of the disclosed gene locus (SEQ ID NO:43), the corresponding proteins can be expressed and purified in a heterologous expression system (e.g., E. coli) and used to raise antibodies (monoclonal or polyclonal) specific for the gougerotin biosynthetic enzymes or proteins, such as GouA, GouB, GouC, GouD, GouE, GouF, GouG, GouH, GouI, GouJ, GouK, GouL, GouM, and/or GouN. Antibodies also may be raised against synthetic peptides derived from the gougerotin amino acid sequences presented herein (SEQ ID NOs:2, 4, 6, 8, 10, 12, 14, 16, 18, 20, 22, 24, 26, 28, 30, 32, 34, 36, 38, 40, and 42). Methods of raising antibodies are well known in the art and are described generally in Harlow and Lane, Antibodies, A Laboratory Manual, Cold Springs Harbor, 1988. Such antibodies can be used to screen an expression library produced from bacteria. For example, this screening will identify the gougerotin orthologs. The selected DNAs can be confirmed by sequencing and enzyme activity assays.

Oligonucleotides derived from a gougerotin gene cluster (SEQ ID NO:43) or nucleic acid sequences encoding ORFs of the gene cluster (SEQ ID NOs:1, 3, 5, 7, 9, 11, 13, 15, 17, 19, 21, 23, 25, 27, 29, 31, 33, 35, 37, 39, and 41), or fragments of these nucleic acid sequences, are encompassed within the scope of the present disclosure. Such oligonucleotides may be used, for example, as probes or primers. In one embodiment, oligonucleotides may comprise a sequence of at least 10 consecutive nucleotides of a gougerotin gene cluster (+/−upstream and downstream flanking regions) or a gougerotin ORF nucleic acid sequence. If these oligonucleotides are used with an in vitro amplification procedure (such as PCR), lengthening the oligonucleotides may enhance amplification specificity. Thus, in other embodiments, oligonucleotide primers comprising at least 15, 20, 25, 30, 35, 40, 45, 50, or more consecutive nucleotides of these sequences may be used. In another example, a primer comprising 30 consecutive nucleotides of a nucleic acid molecule encoding a gougerotin biosynthetic enzyme (such as, for example, SEQ ID NOs: 15 or 21) will anneal to a target sequence, such as a gougerotin gene cluster (+/−upstream and downstream flanking regions) or a gougerotin homolog present in a DNA library from another Streptomyces species (or other gougerotin-producing species), with a higher specificity than a corresponding primer of only 15 nucleotides. In order to obtain greater specificity, probes and primers can be selected that comprise at least 17, 20, 23, 25, 30, 35, 40, 45, 50 or more consecutive nucleotides of the gougerotin gene cluster (+/−upstream and downstream flanking regions) or a gougerotin ORF nucleotide sequence. In particular examples, probes or primers can be at least 100, 250, 500, 600 or 1000 consecutive nucleic acids of a disclosed gougerotin gene cluster (+/−upstream and downstream flanking regions) or a gougerotin ORF sequence.

Oligonucleotides (such as, primers or probes) may be obtained from any region of the disclosed gougerotin gene cluster (+/−upstream and downstream flanking regions) or a gougerotin ORF nucleic acid sequence. By way of example, a gougerotin gene cluster (+/−upstream and downstream flanking regions) or a gougerotin ORF sequence may be apportioned into about halves, thirds or quarters based on sequence length, and the isolated nucleic acid molecules (e.g., oligonucleotides) may be derived from the first or second halves of the molecules, from any of the three thirds, or from any of the four quarters. The nucleic acid sequence of interest also could be divided into smaller regions, e.g., about eighths, sixteenths, twentieths, fiftieths and so forth, with similar effect. Alternatively, it may be divided into regions that encode for conserved domains.

With the provision herein of the gougerotin biosynthetic proteins and corresponding nucleic acid sequences, the creation of variants of these sequences is now enabled. Variant gougerotin biosynthetic enzymes include proteins that differ in amino acid sequence from the disclosed prototype enzymes and still retain the biological activity/function of the prototype proteins as listed in TABLE 1. Variant enzymes may also be stripped of their activity/function producing biosynthetic precursors to, or novel analogs of, gougerotin.

In one embodiment, variant gougerotin biosynthetic proteins include proteins that differ in amino acid sequence from the disclosed gougerotin biosynthetic protein sequences (e.g., SEQ ID NOs:2, 4, 6, 8, 10, 12, 14, 16, 18, 20, 22, 24, 26, 28, 30, 32, 34, 36, 38, 40, and 42) but that share at least 65% amino acid sequence identity with such enzyme sequences. In other embodiments, other variants will share at least 70%, at least 75%, at least 80% at least 85%, at least 90%, at least 91%, at least 92%, at least 93%, at least 94%, at least 95%, at least 96%, at least 97%, at least 98%, or at least 99% amino acid sequence identity. Manipulation of the disclosed gougerotin gene cluster (+/−upstream and downstream flanking regions) and gougerotin ORF nucleotide sequences using standard procedures (e.g., site-directed mutagenesis or PCR), can be used to produce such variants. The simplest modifications involve the substitution of one or more amino acids for amino acids having similar biochemical properties. These so-called conservative substitutions may have minimal impact on the activity of the resultant protein.

### Biosynthetic Production of Gougerotin

Biosynthetic methods for creating gougerotin are useful for its efficient production and can be similarly employed for the production of gougerotin and analogs thereof. Thus, cloning and expression of the gougerotin biosynthetic gene cluster or ORFs therefrom in a heterologous host, such as E. coil or other Streptomyces , spp., can be used to increase production of gougerotin, gougerotin precursor(s), gougerotin intermediate(s), or an enzyme or protein included within the gene cluster. In addition, genetic recombination and domain-exchange constructs permit the creation of gougerotin structures that would be difficult to make using traditional synthetic methodologies.

In an embodiment, a recombinant expression system is selected from prokaryotic hosts. Bacterial cells are available from numerous sources including public sources known to those skilled in the art, such as the American Type Culture Collection (ATCC; Manassas, Va.). Commercial sources of cells used for recombinant protein expression also provide instructions for usage of such cells.

One representative heterologous host system for expression of a gougerotin gene cluster is Streptomyces sp. In specific examples, Streptomyces spp. have been used as artificial hosts to express natural product biosynthetic gene clusters of very large sizes (see, e.g., Stutzman-Engwall and Hutchinson Proc. Natl. Acad. Sci. USA 86: 3135-3139, 1989; Motamedi and Hutchinson Proc. Natl. Acad. Sci. USA 84: 4445-4449, 1987; Grim et al. Gene 151: 1-10 1994; Kao et al. Science 265: 509-512, 1994: and Hopwood et al. Meth. Enzymol. 153: 116-166, 1987). Streptomyces spp. are useful heterologous host systems because they are easily grown, plasmids and cosmids for the expression and/or integration of biosynthetic gene clusters are well characterized, and they house many of the modifying and auxiliary enzymes required to produce functional pathways (Donadio et al. J. Biotechnol. 99:187-198, 2002). A host cell with fragmenting mycelium may exhibit the advantage of keeping viscosity low; further desirable characteristics of a host cell (in addition to the ability to express large amounts of gougerotin) include rapid growth and growth on simple substrates.

Another representative heterologous host system for expression of an gougerotin gene cluster (or one or more of its ORFs) is E. coli. E. coli is an attractive artificial expression system because it is fast-growing and easy to manipulate genetically. Recent advances in E. coli based expression systems have greatly aided efforts to simultaneously express multiple genes in a single host organism. Multiple ORFs from a complex biosynthetic system can now be expressed simultaneously in E. coli.

The choice of expression system will depend, however, on the features desired for the expressed polypeptides. Any transducible cloning vector can be used as a cloning vector for the nucleic acid constructs presently disclosed. If large clusters are to be expressed, it is preferable that phagemids, cosmids, fosmids, P1s, YACs, BACs, PACs, HACs or similar cloning vectors are used for cloning the nucleotide sequences into the host cell. These vectors are advantageous due to their ability to insert and stably propagate larger fragments of DNA, compared to M13 phage and lambda phage, respectively.

In an embodiment, one or more of the disclosed ORFs and/or variants thereof can be inserted into one or more expression vectors, using methods known to those of skill in the art. Vectors are used to introduce gougerotin biosynthesis genes or a gougerotin gene cluster into host cells. Prokaryotic host cells or other host cells with rigid cell walls may be transformed using any method known in the art, including, for example, calcium phosphate precipitation, or electroporation. Representative prokaryote transformation techniques are described in Dower (Genetic Engineering, Principles and Methods 12:275-296, Plenum Publishing Corp., 1990) and Hanahan et al. (Meth. Enzymol. 204:63, 1991), for example. Vectors include one or more control sequences operably linked to the desired ORF. However, the choice of an expression cassette may depend upon the host system selected and features desired for the expressed polypeptide or natural product. Typically, the expression cassette includes a promoter that is functional in the selected host system and can be constitutive or inducible. In an embodiment, the expression cassette includes a promoter, ribosome binding site, a start codon if necessary, and optionally a region encoding a leader peptide in addition to the desired DNA molecule and stop codon. In addition, a 3′ terminal region (translation and/or transcription terminator) can be included within the cassette. The ORF constituted in the DNA molecule may be solely controlled by the promoter so that transcription and translation occur in the host cell. Promoter-encoding regions are well known and available to those of skill in the art. Examples of promoters can include bacterial promoters (such as those derived from sugar metabolizing enzymes, such as galactose, lactose and maltose), promoter sequences derived from biosynthetic enzymes such as tryptophan, the beta-lactamase promoter system, bacteriophage lambda PL and TF and viral promoters.

The presence of additional regulatory sequences within the expression cassette may be desirable to allow for regulation of expression of the one or more ORFs relative to the growth of the host cell. These regulatory sequences are well known in the art. Examples of regulatory sequences include sequences that turn gene expression on or off in response to chemical or physical stimulus as well as enhancer sequences. In addition to the regulatory sequences, selectable markers can be included to assist in selection of transformed cells. For example, genes that confer antibiotic resistance or sensitivity to the plasmid may be used as selectable markers.

It is contemplated that the gougerotin gene cluster or one or more gougerotin ORFs of interest can be cloned into one or more recombinant vectors as individual cassettes, with separate control elements, or under the control of a single control element (e.g., a promoter). In an embodiment, the ORFs include two or more restriction sites to allow for the easy deletion and insertion of other open reading frames so that hybrid synthetic pathways can be generated. The design and use of such restriction sites is well known in the art and can be carried out by using techniques described above such as PCR or site-directed mutagenesis. Proteins expressed by the transformed cells can be recovered according to standard methods well known to those of skill in the art. For example, proteins can be expressed with a convenient tag to facilitate isolation. Further, the resulting polypeptide can be purified by affinity chromatography by using a ligand that binds to the polypeptide.

It is further contemplated that various gougerotin ORFs, gene cluster, or gougerotin proteins of interest may be produced by utilizing fermentation conditions as previously described for the production of gougerotin. After production, the compounds can be purified and/or analyzed by methods well known to one of skill in the art including, for example, high-pressure liquid chromatography (HPLC).

The present invention also encompasses methods of treating a plant to control plant pests and diseases by administering to a plant or a plant part, such as a leaf, stem, flowers, fruit, root, or seed or by applying to a locus on which plant or plant parts grow, such as soil, one or more of a gougerotin containing fermentation broth of Streptomyces, the Streptomyces microflavus strain NRRL B-50550 or a phytophagous-miticidal mutant strain thereof or cell-free preparations thereof or metabolites thereof.

As used herein, the term “plant” refers to any living organism belonging to the kingdom Plantae (i.e., any genus/species in the Plant Kingdom). This includes familiar organisms such as but not limited to trees, herbs, bushes, grasses, vines, ferns, mosses and green algae. The term refers to both monocotyledonous plants, also called monocots, and dicotyledonous plants, also called dicots. The plant is in some embodiments of economic importance. In some embodiments the plant is a men-grown plant, for instance a cultivated plant, which may be an agricultural, a silvicultural or a horticultural plant. Examples of particular plants include but are not limited to corn, potatoes, roses, apple trees, sunflowers, wheat, rice, bananas, tomatoes, opo, pumpkins, squash, beans (e.g., lima beans), lettuce, cabbage, oak trees, guzmania, geraniums, hibiscus, clematis, poinsettias, sugarcane, taro, duck weed, pine trees, Kentucky blue grass, zoysia, coconut trees, brassica leafy vegetables (e.g., broccoli, broccoli raab, Brussels sprouts, cabbage, Chinese cabbage (Bok Choy and Napa), cauliflower, cavalo, collards, kale, kohlrabi, mustard greens, rape greens, and other brassica leafy vegetable crops), bulb vegetables (e.g., garlic, leek, onion (dry bulb, green, and Welch), shallot, and other bulb vegetable crops), citrus fruits (e.g., grapefruit, lemon, lime, orange, tangerine, citrus hybrids, pummelo, and other citrus fruit crops), cucurbit vegetables (e.g., cucumber, citron melon, edible gourds, gherkin, muskmelons (including hybrids and/or cultivars of cucumis melons), water-melon, cantaloupe, and other cucurbit vegetable crops), fruiting vegetables (including eggplant, ground cherry, pepino, pepper, tomato, tomatillo, and other fruiting vegetable crops), grape, leafy vegetables (e.g., romaine), root/tuber and corm vegetables (e.g., potato), lentils, alfalfa sprouts, clover and tree nuts (almond, pecan, pistachio, and walnut), berries (e.g., tomatoes, barberries, currants, elderberries, gooseberries, honeysuckles, mayapples, nannyberries, Oregon-grapes, see-buckthorns, hackberries, bearberries, lingonberries, strawberries, sea grapes, blackberries, cloudberries, loganberries, raspberries, salmonberries, thimbleberries, and wineberries), cereal crops (e.g., corn, rice, wheat, barley, sorghum, millets, oats, ryes, triticales, buckwheats, fonio, and quinoa), pome fruit (e.g., apples, pears), stone fruits (e.g., coffees, jujubes, mangos, olives, coconuts, oil palms, pistachios, almonds, apricots, cherries, damsons, nectarines, peaches and plums), vine (e.g., table grapes, wine grapes), fibber crops (e.g., hemp, cotton), ornamentals, to name a few. The plant may, in some embodiments, be a household/domestic plant, a greenhouse plant, an agricultural plant, or a horticultural plant. As already indicated above, in some embodiments the plant may a hardwood such as one of acacia, eucalyptus, hornbeam, beech, mahogany, walnut, oak, ash, willow, hickory, birch, chestnut, poplar, alder, maple, sycamore, ginkgo, a palm tree and sweet gum. In some embodiments the plant may be a conifer such as a cypress, a Douglas fir, a fir, a sequoia, a hemlock, a cedar, a juniper, a larch, a pine, a redwood, spruce and yew. In some embodiments the plant may be a fruit bearing woody plant such as apple, plum, pear, banana, orange, kiwi, lemon, cherry, grapevine, papaya, peanut, and fig. In some embodiments the plant may be a woody plant such as cotton, bamboo and a rubber plant. The plant may in some embodiments be an agricultural, a silvicultural and/or an ornamental plant, i.e. a plant which is commonly used in gardening, e.g., in parks, gardens and on balconies. Examples are turf, geranium, pelargonia, petunia, begonia, and fuchsia, to name just a few among the vast number of ornamentals. The term “plant” is also intended to include any plant propagules.

The term “plant” generally includes a plant that has been modified by one or more of breeding, mutagenesis and genetic engineering. Genetic engineering refers to the use of recombinant DNA techniques. Recombinant DNA techniques allow modifications which cannot readily be obtained by cross breeding under natural circumstances, mutations or natural recombination. In some embodiments a plant obtained by genetic engineering may be a transgenic plant.

As used herein, the term “plant part” refers to any part of a plant including but not limited to the shoot, root, stem, seeds, stipules, leaves, petals, flowers, ovules, bracts, branches, petioles, internodes, bark, wood, tubers, pubescence, tillers, rhizomes, fronds, blades, pollen, stamen, microspores, fruit and seed. The two main parts of plants grown in typical media employed in the art, such as soil, are often referred to as the “above-ground” part, also often referred to as the “shoots”, and the “below-ground” part, also often referred to as the “roots”.

In a method according to the invention a composition containing Streptomyces microflavus NRRL B-50550 or a phytophagous-miticidal mutant strain thereof can be applied to any plant or any part of any plant grown in any type of media used to grow plants (e.g., soil, vermiculite, shredded cardboard, and water) or applied to plants or the parts of plants grown aerially, such as orchids or staghorn ferns. The composition may for instance be applied by spraying, atomizing, vaporizing, scattering, dusting, watering, squirting, sprinkling, pouring or fumigating. As already indicated above, application may be carried out at any desired location where the plant of interest is positioned, such as agricultural, horticultural, forest, plantation, orchard, nursery, organically grown crops, turfgrass and urban environments.

Compositions of the present invention can be obtained by culturing Streptomyces microflavus NRRL B-50550 or mutants derived from it using conventional large-scale microbial fermentation processes, such as submerged fermentation, solid state fermentation or liquid surface culture, including the methods described, for example, in U.S. Pat. No. 3,849,398, British Patent No. GB 1 507 193, Toshiko Kanzaki et al., Journal of Antibiotics, Ser. A, Vol. 15, No. 2, June. 1961, pages 93 to 97, or Toru Ikeuchi et al., Journal of Antibiotics, (September 1972), pages 548 to 550. Fermentation is configured to obtain high levels of live biomass, particularly spores, and desirable secondary metabolites in the fermentation vessels. Specific fermentation methods that are suitable for the strain of the present invention to achieve high levels of sporulation, cfu (colony forming units), and secondary metabolites are described in the Examples section.

The bacterial cells, spores and metabolites in culture broth resulting from fermentation (the “whole broth” or “fermentation broth”) may be used directly or concentrated by conventional industrial methods, such as centrifugation, filtration, and evaporation, or processed into dry powder and granules by spray drying, drum drying and freeze drying, for example.

The terms “whole broth” and “fermentation broth,” as used herein, refer to the culture broth resulting from fermentation before any downstream treatment. The whole broth encompasses the microorganism (e.g., Streptomyces microflavus NRRL B-50550 or a phytophagous-miticidal mutant strain thereof) and its component parts, unused raw substrates, and metabolites produced by the microorganism during fermentation. The term “broth concentrate,” as used herein, refers to whole broth (fermentation broth) that has been concentrated by conventional industrial methods, as described above, but remains in liquid form. The term “fermentation solid,” as used herein, refers to dried fermentation broth. The term “fermentation product,” as used herein, refers to whole broth, broth concentrate and/or fermentation solids. Compositions of the present invention include fermentation products. In some embodiments, the concentrated fermentation broth is washed, for example, via a diafiltration process, to remove residual fermentation broth and metabolites.

In one embodiment, the fermentation broth or broth concentrate can be formulated into liquid suspension, liquid concentrate, or emulsion concentrate with the addition of stabilization agents, preservatives, adjuvants, and/or colorants.

In another embodiment, the fermentation broth or broth concentrate can be dried with or without the addition of carriers, inerts, or additives using conventional drying processes or methods such as spray drying, freeze drying, tray drying, fluidized-bed drying, drum drying, or evaporation.

In a further embodiment, the resulting dry products may be further processed, such as by miffing or granulation, with or without the addition of inerts or additives to achieve specific particle sizes or physical formats or physical properties desirable for agricultural applications.

In addition to the use of whole broth or broth concentrate, cell-free preparations of fermentation broth of the novel variants and strains of Streptomyces of the present invention can be obtained by any means known in the art, such as extraction, centrifugation and/or filtration of fermentation broth. Those of skill in the art will appreciate that so-called cell-free preparations may not be devoid of cells but rather are largely cell-free or essentially cell-free, depending on the technique used (e.g., speed of centrifugation) to remove the cells. The resulting cell-free preparation may be dried and/or formulated with components that aid in its application. Concentration methods and drying techniques described above for fermentation broth are also applicable to cell-free preparations.

Compositions of the present invention may include formulation ingredients added to compositions comprising cells, cell-free preparations or metabolites to improve efficacy, stability, and physical properties, usability and/or to facilitate processing, packaging and end-use application. Such formulation ingredients may include carriers, inerts, stabilization agents, preservatives, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the ingredient is a binder, adjuvant, or adhesive that facilitates adherence of the composition to a plant part, such as leaves, seeds, or roots. See, for example, Taylor, A. G., et al., “Concepts and Technologies of Selected Seed Treatments” Annu. Rev. Phytopathol. 28: 321-339 (1990). The stabilization agents may include anti-caking agents, anti-oxidation agents, desiccants, protectants or preservatives. The nutrients may include carbon, nitrogen, and phosphors sources such as sugars, polysaccharides, oil, proteins, amino acids, fatty acids and phosphates. The physical property modifiers may include bulking agents, wetting agents, thickeners, pH modifiers, rheology modifiers, dispersants, adjuvants, surfactants, antifreeze agents or colorants. In some embodiments, the composition comprising cells, cell-free preparation or metabolites produced by fermentation can be used directly with or without water as the diluent without any other formulation preparation. In some embodiments, the formulation inerts are added after concentrating fermentation broth and during and/or after drying.

In some embodiments the compositions of the present invention are used to treat a wide variety of agricultural and/or horticultural crops, including those grown for seed, produce, landscaping and those grown for seed production. Representative plants that can be treated using the compositions of the present invention include but are not limited to the following: brassica, bulb vegetables, cereal grains, citrus, cotton, cucurbits, fruiting vegetables, leafy vegetables, legumes, oil seed crops, peanut, pome fruit, root vegetables, tuber vegetables, corm vegetables, stone fruit, tobacco, strawberry and other berries, and various ornamentals.

The compositions of the present invention may be administered as a foliar spray, as a soil treatment, and/or as a seed treatment/dressing. When used as a foliar treatment, in one embodiment, about 1/16 to about 5 gallons of whole broth are applied per acre. When used as a soil treatment, in one embodiment, about 1 to about 5 gallons of whole broth are applied per acre. When used for seed treatment about 1/32 to about ¼ gallons of whole broth are applied per acre. For seed treatment, the end-use formulation contains at least 1×10⁸ colony forming units per gram.

### Deposit Information

A sample of the Streptomyces microflavus strain of the invention has been deposited with the Agricultural Research Service Culture Collection located at the National Center for Agricultural Utilization Research, Agricultural Research Service, U.S. Department of Agriculture, 1815 North University Street, Peoria, Ill. 61604 under the Budapest Treaty and has been assigned the following depository designation: NRRL B-50550.

The following examples are given for purely illustrative and non-limiting purposes of the present invention.

### EXAMPLES

### Example 1

### Selection of Streptomyces microflavus NRRL B-50550

Strains were taken from an internal collection of strains and initial screening tests were conducted to determine efficacy of potential candidates strain against two-spotted spider mites (“TSSM”), which are a model organism commonly used to screen for general miticidal activity. Microorganisms were selected initially for properties that favor laboratory or artificial cultivation, such as variants that grow rapidly on an agar plate. Culture stocks of the selected strains were grown in suitable media for the respective strain, such as the Medium 1 and Medium 2 described in Example 2. The resulting fermentation products (whole broths) were diluted to a 25% solution using water and 0.03% of the surfactant BREAK-THRU FIRST CHOICE®. Thereafter, 8 ml of the diluted fermentation products were applied to run-off to the top and bottom of lima bean leaves of two plants (the lima bean plants were 1 to 1.5 weeks old). After such treatment, plants were infested on the same day with 50-100 TSSM and left in the greenhouse for five days. On the fifth day plants were assessed for presence of mites and eggs on a scale of 1 to 4. The miticide Avid® (Syngenta) was used as positive control. For mites and eggs, 1 indicates 100% mortality, 1.5 indicates 90% to 95% mortality, 2.0 represents 75% to 90% mortality; 2.5 represents 40% to 55% mortality; 3.0 represents 20% to 35% mortality and 4.0 represents 0% to 10% mortality. Besides NRRL B-50550, other Streptomyes strains and some Bacillus strains were found to be active against mites.

For further selection, amongst other activities, the UV stability and translaminar activity of the screened strains was examined since an acaracide should be stable to UV light and possess translaminar activity in order to be effective in field applications.

For assessment of the UV stability the above-described 25% dilutions of the fermentation products were sprayed on the upper surface of lima bean plants. After such treatment, plants were infested on the same day with 50-100 TSSM, exposed to UV light for 24 hrs and left in the greenhouse for five days. The mites were confined to the adaxial (upper) surface of the leaves by means of a Vaseline ring which was applied to the leaf and served as an impassable boundary to the mites. On the fifth day plants were assessed for presence of mites and eggs on a scale of 1 to 4, as described above. The miticides Avid® (Syngenta) and Oberon® (Bayer CropScience AG) were used as controls. Results are shown in FIG. 1. The fermentation product of the strain NRRL B-50550 showed the best UV stability of all strains tested.

For assessment of the translaminar activity the strains were cultured as described above and the resulting whole broth was diluted using water and 0.35% surfactant and applied to run-off to the lower surface of lima bean leaves on two plants. The upper surface of the treated leaves was infested one day after treatment with 50-100 TSSM, which were placed on the upper surface of the leaves and contained using a Vaseline ring/physical barrier as described above. On the sixth day plants were assessed for presence of mites and eggs on the above-described scale of 1 to 4. The miticides Avid® (Syngenta) and Oberon® (Bayer CropScience AG) were used as controls. Results are shown in FIG. 2. The fermentation product of the strain NRRL B-50550 showed the best translaminar activity of all strains tested.

### Example 2

### Supplement Media with Cytosine

Without wishing to be bound by theory, Applicant postulates that the availability of cytosine is critical to the production of gougerotin, as shown by the hypothetical synthetic pathway of FIG. 2. Thus, with increasing levels of cytosine provided in a culture medium, the amount of gougerotin obtained should also increase. Accordingly, Applicant tested gougerotin production by Streptomyces microflavus B-50550 in media to which cytosine, thymine and/or uracil were added. Specifically, Streptomyces microflavus B-50550 was grown in a media composed of 10.0 g/L starch, 15.0 g/L glucose, 10.0 g/L yeast extract, 10.0 g/L casein hydrolysate (or 10.0 g/l soy peptone), 2.0 g/L CaCO₃ and cytosine, uracil and/or thymine, each at a concentration of 0.50 g/L, in 2 L shake flasks at 20-30 C for 6 days. Results are shown in the table below.

TABLE 2

| | Cytosine | Uracil | Thymine | Gougerotin at | Gougerotin at |
|---|---|---|---|---|---|
| Run | (g/L) | (g/L) | (g/L) | Day 4 (g/L) | Day 6 (g/L) |
| 1 | 0.50 | 0.50 | 0 | 1.6 | 2.4 |
| 2 | 0.50 | 0 | 0.50 | 1.4 | 2.2 |
| 3 | 0.50 | 0.50 | 0.50 | 1.2 | 2.1 |
| 4 | 0 | 0.50 | 0.50 | 1.3 | 1.7 |
| 5 | 0.50 | 0 | 0 | 1.6 | 2.4 |
| 6 | 0 | 0.50 | 0 | 1.4 | 2.0 |
| 7 | 0 | 0 | 0.50 | 1.1 | 1.4 |
| 8 | 0 | 0 | 0 | 1.2 | 1.5 |

Applicant also intends to test gougerotin production by Streptomyces microflavus in media with the addition of varying levels of cytosine. Finally, Applicant intends to investigate other food sources for fermentation with high cytosine sources (e.g., high nucleotide yeast extract).

### Example 3

### Knocking Out Gougerotin Gene Cluster in NRRLB-50550 to Confirm its Function

Studies will be conducted to confirm that the putative gene cluster is responsible for the gougerotin expression. Three constructs containing the aminotransferase gene (ORF 4258), similar to the cytosinine synthase of blasticidin, the cytosylglucuronic acid synthase gene (ORF 4261), and a dehydrogenase (ORF 4253), which might be involved in the production of UDP-glucuronic acid plus 300 nucleotides upstream of the coding region will be generated from NRRLB-50550 genomic DNA. Without wishing to be bound by theory, Applicant postulates that the aminotransferase gene (ORF 4258) and the cytosylglucuronic acid synthase gene (ORF 4261) are required relatively early in the gougoritin biosynthetic pathway. Because ORF 4258 and ORF 4261 are close to one another in the genome, and also because they are located in the middle of the gene cluster, the dehydrogenase gene (ORF 4253), which might be involved in the production of UDP-glucuronic acid. Applicant postulates that this enzyme should also be required early in the pathway.

Primers will be used that contain restriction enzyme sites for subcloning the DNA fragments into a plasmid vector named pAH77 and designed to allow for conjugation in NRRLB-50550 cells and double recombination in NRRLB-50550 genomic DNA. The plasmid pAH77 will be transformed into E. coli strain ET12567/pUZ8002 which will facilitate the movement of DNA cloned in the plasmid vector into Streptomyces NRRLB-50550. (Strain ET12567 is a methylation-deficient host, and used to circumvent methyl-specific restriction systems possessed by some Streptomyces strains). The movement of DNA into NRRLB-50550 occurs by conjugation with the desired DNA construct inserted between two domains which are homologous to locations in the Streptomyces genome.

To allow for natural competence to occur, E. coli strain ET12567 containing circular plasmid DNA pUZ8002 with either i) the ORF 4258 gene or ii) the ORF 426 gene or iii) the ORF 4253 gene will be grown overnight, diluted 1:100 in fresh LB (Difco bacto tryptone, 10g/L; Difco yeast extract, 5g/L; NaCl, 5 g/L; Glucose, 1 g/L) plus antibiotic selection (Chloramphenicol=25 μg/m, Kanamycin=25 μg/m, Apramycin=100 μg/ml) and grown at 37° C. to an OD₆₀₀ of 0.4-0.6. The cells will be washed and resuspended in 0.1 volume of LB. For each conjugation, approximately 10⁸ of NRRLB-50550 spores will be added to 500 μl 2xYT Broth (Difco bacto tryptone, 16 g/L; Difco bacto yeast extract, 10 g/L; NaCl, 5 g/L), heat shocked at 50° C. for 10 min then allowed to cool. 500 μl of E. coli cells will be added to 0.5 ml heat-shocked NRRLB-50550 Streptomyces spores, mixed and spinned briefly for 5 minutes at 4200 rpm. The supernatant will be poured off and the pellet resuspended in the residual liquid.

The cells will be plated out on MS agar+10 mM MgCl₂ (Agar 20 g; Mannitol 20 g; Soya flour 20 g) and incubated at 30° C. for 16-20 hours. The plates will be overlayed with 1 ml of water containing 0.5 mg nalidixic acid and the appropriate plasmid selection (e.g. 1 mg apramycin for those vectors conferring apramycin resistance), using a spreader to distribute the antibiotic solution evenly. The plates will be placed at 30° C.

The potential Streptomyces NRRLB-50550 exconjugants (in theory, Streptomyces NRRLB-50550 colonies that grow on the MS plates with apramycin selection should have acquired the plasmid containing either of the NRRLB-50550 gene (either orf4258, orf4261 or orf4253 gene) and the apramycin resistance gene) will be plated onto selective media containing nalidixic acid (25 μg/ml).

NRRLB-50550, NRRLB-50550:: orf4258, NRRLB-50550:: orf4261 and NRRLB-50550:: orf4253 cultures will be grown side by side in shake flasks for comparison by analytical chemistry. Two conjugate colonies will be chosen and streaked in order to obtain a lawn of cells. One plug of each conjugate and one plug of NRRLB-50550 will be used to inoculate 12 mls of medium consisting of 10.0 g/L starch, 15.0 g/L glucose, 10.0 g/L yeast extract, 10.0 g/L casein hydrolysate (or 10.0 g/l soy peptone), and 2.0 g/L CaCO₃in a 50 ml centrifuge tube. Tube cultures will be grown for 48 hours at 28° C., 180 rpm. After 48 hours, 2.5 mls of tube culture will used to inoculate 50ml of the above-described medium in a 250 ml baffled flask. Flasks will be grown for 7 days at 28° C., 180 rpm and analyzed for gougerotin production.

Applicant hypothesizes that NRRLB-50550 will produce gougerotin while NRRLB-50550:: orf4258, NRRLB-50550:: orf4261 and NRRLB-50550:: orf4253 will not.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

We claim:

1. A nucleic acid sequence having at least 70% sequence identity to the nucleotide sequence of SEQ ID NO:43, operably linked to at least one exogenous and/or heterologous regulatory element for directing expression of said nucleotide sequence.

2. The nucleic acid sequence of claim 1, wherein the isolated gene cluster is isolated from S. microflavus, S. griseus, S. anulatus, S. fimicarius, S. parvus, S. lavendulae, and S. alboviridis.

3. A host cell comprising the nucleic acid sequence of claim 1.

4. A method for producing a gougerotin or gougerotin analog, comprising: cultivating a gougerotin or gougerotin-producing bacterium of the Streptomyces family in a medium to produce and excrete said gougerotin or gougerotin analog into the medium, and collecting said gougerotin or gougerotin analog from the medium, wherein said bacterium has been modified to enhance expression of genes of the nucleic acid sequence of claim 1.

5. The method of claim 4, wherein the bacterium is is selected from the group consisting of S. microflavus, S. griseus, S. anulatus, S. fimicarius, S. parvus, S. lavendulae, and S. alboviridis.

6. The method of claim 4, wherein the bacterium is S. microflavus.

7. An expression vector comprising a gene encoding a polypeptide subunit selected from the nucleic acid sequence of claim 1.

8. A host cell comprising the vector of claim 7.

9. A method for preparing a gougerotin or gougerotin analog, comprising the following steps:
a) constructing a recombinant expression vector containing the nucleic acid sequence of claim 1;
b) transforming a host cell with the expression vector containing the nucleic acid sequence of step a) to produce a transformant;
c) culturing the transformant of step b); and
d) isolating and purifying said gougerotin or gougerotin analog from the culture product of the transformant of step c).

10. A transgenic prokaryotic cell comprising a nucleic acid sequence encoding an amino acid sequence having at least 70% sequence identity to at least one amino acid sequence selected from the group consisting of SEQ ID NO:2, SEQ ID NO:4, SEQ ID NO:6, SEQ ID NO:10, SEQ ID NO:12, SEQ ID NO:14, SEQ ID NO:16, SEQ ID NO:18, SEQ ID NO:22, SEQ ID NO:24, SEQ ID NO:26, SEQ ID NO:28, SEQ ID NO:30, and SEQ ID NO:42.

11. A nucleic acid sequence having at least 70% sequence identity to the nucleotide sequence of SEQ ID NO:1, SEQ ID NO:3, SEQ ID NO:5, SEQ ID NO:9, SEQ ID NO:11, SEQ ID NO:13, SEQ ID NO:15, SEQ ID NO:17, SEQ ID NO:21, SEQ ID NO:23, SEQ ID NO:25, SEQ ID NO:27, SEQ ID NO:29, SEQ ID NO:41, or a combination of two or more of said nucleotide sequences, operably linked to at least one exogenous and/or heterologous promoter for directing expression of said sequence or of said combination.

12. A nucleic acid sequence having at least 70% sequence identity to a nucleic acid sequence selected from the group consisting of SEQ ID NO:1, SEQ ID NO:3, SEQ ID NO:5, SEQ ID NO:9, SEQ ID NO:11, SEQ ID NO:13, SEQ ID NO:15, SEQ ID NO:17, SEQ ID NO:21, SEQ ID NO:23, SEQ ID NO:25, SEQ ID NO:27, SEQ ID NO:29, and SEQ ID NO:41, further comprising a nucleic acid sequence comprising an exogenous restriction enzyme cleavage site.


SEQUENCE LISTING (43 sequences)

SEQ ID NO: 1 - DNA, 426 bp, Streptomyces microflavus
  misc_feature (1)..(426): orf4251; Methyltransferase domain family; GouA
gtgatcccctactccacgttccagttactccggaccgtcgaggaccgggagcgagcactgacggaagccgtacgcgccatgaggccaggtgccacagtccacatcgatgtcagcagcaacttcgacctgcgggagagcagcgactggcagcgggtactgtccgccccctgcgaagaagtgagcaggcagcccgtcgaggaatgggaacgaacaaggtcccacccagaccacatcctcatcgacaaaaccttccgaacagacagaagcgttctcctccggttcacggagcattggacccacctgcgggcactcgatctcgaagccgctctactgcgtgccgggttgattctggaacaggtcgaccacggctacggcgggggcgcatcaccccaccgccggatctaccacgcccgccgacctctctga

SEQ ID NO: 2 - protein, 141 aa, Streptomyces microflavus
  MISC_FEATURE (1)..(141): orf4251; Methyltransferase domain family; GouA
MIPYSTFQLLRTVEDRERALTEAVRAMRPGATVHIDVSSNFDLRESSDWQRVLSAPCEEVSRQPVEEWERTRSHPDHILIDKTFRTDRSVLLRFTEHWTHLRALDLEAALLRAGLILEQVDHGYGGGASPHRRIYHARRPL

SEQ ID NO: 3 - DNA, 462 bp, Streptomyces microflavus
  misc_feature (1)..(462): orf4252; major facilitator transporter; GouB
gtggcgggcctggccaacgcgtgtttcgccctggcggcgatcggcagtggagtgctcgtctccggaggatcgctgggcaactgggtgcgcagccacacaccaggagtcctcatcgccggtttcgctgtgcagacggtgttcggacttaccgcgagtcagaccgtcttagccgttgccctctacacgctggtcggtgtgctcagcggcggggacaccgtgctgcagagcgaggtgcaggatcgctggcgaagtgtcgggtcctcccaggcgttcgccattttcggtgcggtgtccggaccttcccaactggtcggctccttggtcatcgcttttctgctcctgcacttctcgatcaccgctgtctacgtcggaacgattgtgattctgggattcggttcggcgctccttctggtcatggcgagagcgaagtcgatcaatcccgtggaagaagtcgtcccgtaa

SEQ ID NO: 4 - protein, 153 aa, Streptomyces microflavus
  MISC_FEATURE (1)..(153): orf4252; major facilitator transporter; GouB
MAGLANACFALAAIGSGVLVSGGSLGNWVRSHTPGVLIAGFAVQTVFGLTASQTVLAVALYTLVGVLSGGDTVLQSEVQDRWRSVGSSQAFAIFGAVSGPSQLVGSLVIAFLLLHFSITAVYVGTIVILGFGSALLLVMARAKSINPVEEVVP

SEQ ID NO: 5 - DNA, 516 bp, Streptomyces microflavus
  misc_feature (1)..(516): orf4253; 3-hydroxyisobutyrate dehydrogenase; GouC
gtgcgggacctgcacacccggatgcgggacacccacggacacacgctggtggatgccgcgctcagccgccgcggcggtgttgtgcgcgagggttcgctgtccctgttcgtcggggcaggagacgaggccttcgccgtagcccggccggtcttcgaccgctacgccgacaacgtcgtccacgccggagacgtcggtgccggcatgacggtcaagctttgcaacaactggctgctctacgcgaaccggcactccgcactccaggccatccgaacgggccgtcagctcggcgtggatccggctgtgctcacggacgcgctcgcctcttccaccggatccagctgggcactgtcccactactccgacctggacgaagccatcgtcaccgggcggggcgcaccagcggtcatccgggacaggacagcttcggagcttggcatggcgaagcagatggcggcacaggacggtgaggtgcccaccagcctccaggagaccttcgcccttctggacgtgatgtag

SEQ ID NO: 6 - protein, 171 aa, Streptomyces microflavus
  MISC_FEATURE (1)..(171): orf4253; 3-hydroxyisobutyrate dehydrogenase; GouC
MRDLHTRMRDTHGHTLVDAALSRRGGVVREGSLSLFVGAGDEAFAVARPVFDRYADNVVHAGDVGAGMTVKLCNNWLLYANRHSALQAIRTGRQLGVDPAVLTDALASSTGSSWALSHYSDLDEAIVTGRGAPAVIRDRTASELGMAKQMAAQDGEVPTSLQETFALLDVM

SEQ ID NO: 7 - DNA, 357 bp, Streptomyces microflavus
  misc_feature (1)..(357): orf4254; hypothetical protein
gtgacggtcccgcaggacgaacgcctgtccgcgcaggacgccttcggcgtccaccgccgcgagacagcggaccgccccttcagagcggcggaggcgcagtccggcggctctccgttccgccacagccgcctcggactggccaacgacggcgcgagcgaagtcgaactcacacggatctgcggcctccacacgcagagcgcgccccgcagcccgcagatggttgcggacggtggggctgtaaccccctcgggggtcacagccctcacgtatgtccagcatgcgggtgtgtcggcacgccacgtctccacccacctcagcgagtgcagctacttcgccgaaacacggatccatgggtaa

SEQ ID NO: 8 - protein, 118 aa, Streptomyces microflavus
  MISC_FEATURE (1)..(118): orf4254; hypothetical protein
MTVPQDERLSAQDAFGVHRRETADRPFRAAEAQSGGSPFRHSRLGLANDGASEVELTRICGLHTQSAPRSPQMVADGGAVTPSGVTALTYVQHAGVSARHVSTHLSECSYFAETRIHG

SEQ ID NO: 9 - DNA, 141 bp, Streptomyces microflavus
  misc_feature (1)..(141): orf4255; CoA-binding domain-containing protein; GouD
gtgaactctttcgattttgaaggaagtgtcgttccgggcatcgaccgcttcatggctggcttcggcgctcaggccgtcggttacgggcagttccgctggcaacaggattggcgggaagaccctgccgggaggttcgggtga

SEQ ID NO: 10 - protein, 46 aa, Streptomyces microflavus
  MISC_FEATURE (1)..(46): orf4255; CoA-binding domain-containing protein; GouD
MNSFDFEGSVVPGIDRFMAGFGAQAVGYGQFRWQQDWREDPAGRFG

SEQ ID NO: 11 - DNA, 915 bp, Streptomyces microflavus
  misc_feature (1)..(915): orf4256; hypothetical protein; GouE
gtgcagcagtcgcccgcgtacgcccgtgcggtcggcgagtcggggcggtacgtccttgtggtgatgggccaccggggggtgttcccgctgcagctcggctcggacgtgtgcacagcgctcggcggcgaccggccgctcctcgcggacggcaccggccccctgcccctggttcctctggtggccgcggccgcagaggccaccgggctccccgtctacctccctctggtggacgcttcgctcgccgaggcaggagttgtggaggcgttcagcgtgtgggagcggccacccaattcactgatcgactggtcgctcgacggcgccgacctatgggatcgcgtaaccgagcgcggtggttcacagtggagcaggaaacagcggctcatcgagcgggacggcctgattctgtccttcggccggtcgggcgaggcggccgccgaggaggtcctgcggatcgatgaccgctcgtggaagtctgcccacaggcagaacatgcgtgcgcgagagggccaggacaagttgtacgccgggctgatcgggtcaggggtgctcacggctacgttcctgcgggacggcgaccgctctgtcgccttccggcttgactcccgcgtcaaaggccgcctgacctgcttgaagtggtcgtatgacgagtcttaccggcgatactcacccggtgtccatttgctcacgcaggggctgcgccaggagtggtgcgggcgcggcatagaggtggtcgatctgcacgggagtcccgactcactgaaggacctgctgtgcaccgaccgggtgagccgcgtggacctctggtacggcgacccgctggccggcgcgcgccgtgcggccgagcggaccggcttcgacaggcggatgagggcggtccgtgacggtgggaaggggttacgccatgccttcgagtga

SEQ ID NO: 12 - protein, 304 aa, Streptomyces microflavus
  MISC_FEATURE (1)..(304): orf4256; hypothetical protein; GouE
MQQSPAYARAVGESGRYVLVVMGHRGVFPLQLGSDVCTALGGDRPLLADGTGPLPLVPLVAAAAEATGLPVYLPLVDASLAEAGVVEAFSVWERPPNSLIDWSLDGADLWDRVTERGGSQWSRKQRLIERDGLILSFGRSGEAAAEEVLRIDDRSWKSAHRQNMRAREGQDKLYAGLIGSGVLTATFLRDGDRSVAFRLDSRVKGRLTCLKWSYDESYRRYSPGVHLLTQGLRQEWCGRGIEVVDLHGSPDSLKDLLCTDRVSRVDLWYGDPLAGARRAAERTGFDRRMRAVRDGGKGLRHAFE

SEQ ID NO: 13 - DNA, 981 bp, Streptomyces microflavus
  misc_feature (1)..(981): orf4257; DegT/DnrJ/EryC1/StrS aminotransferase; GouF
gtggaacggcccgacagaggtgccacgggcttcgccggtcccgccgggtacgtcaccgtcggtgaagccgtcgcggaactgacccgtcgcatggcccagcccgcggtcgagttcaccgcctccggtaccgccgctctcgaagcggcactggaggtgctgggcatcggacggggcgatgaagtggttgtgcctgacgtgggatgtcactcggtggccgccgccgtcgtgcgacggggagcgatcccggtgttcacgggagtgggggaggccctgacgctcgatccccggggggtttccctggcctgcggcccacgcacccgcgctgtcgttgccgtgcatcagtacggactgccctgtgatgtgcccggcatcatggaggcggtggggccggacatcccggtgatcgaggatgtcgcccagacgtgggggtcggcggtgggcggtgccccggcggggtcgctcgggaccatcgccgtcatttctttcggttcgaccaagccggtggcgctcggcgcgggcggggcgctcttcgggccggcctcactgatcggcggagcggtctcccgcggcgatggagcggaccggcagctgcttcgccctcccagcgccgctcggttccccgcccccctgctcgcccatcttcccaaagccttggaaagggctgaccggctgctggccttgcgtcgggcagcggtggaaacctttctccgtgggccgctggcccaggagttacgtctgcctccgacgccacccggctcctcctccggatggacccgcacccctctgtatccgatcgcccccgcgacctcggtcacggccgaacacatggagcggctggaggcgtgtcacggcccggttcagcgcatgcacgcgacgccgccgtcggcgctgccgatgttccgcggaagcacaacgcgtgtgacaggcggcggccgtcggctcaccgaacccctactcgtgaagatgggatcaccacgatga

SEQ ID NO: 14 - protein, 326 aa, Streptomyces microflavus
  MISC_FEATURE (1)..(326): orf4257; DegT/DnrJ/EryC1/StrS aminotransferase; GouF
MERPDRGATGFAGPAGYVTVGEAVAELTRRMAQPAVEFTASGTAALEAALEVLGIGRGDEVVVPDVGCHSVAAAVVRRGAIPVFTGVGEALTLDPRGVSLACGPRTRAVVAVHQYGLPCDVPGIMEAVGPDIPVIEDVAQTWGSAVGGAPAGSLGTIAVISFGSTKPVALGAGGALFGPASLIGGAVSRGDGADRQLLRPPSAARFPAPLLAHLPKALERADRLLALRRAAVETFLRGPLAQELRLPPTPPGSSSGWTRTPLYPIAPATSVTAEHMERLEACHGPVQRMHATPPSALPMFRGSTTRVTGGGRRLTEPLLVKMGSPR

SEQ ID NO: 15 - DNA, 1194 bp, Streptomyces microflavus
  misc_feature (1)..(1194): orf4258; DegT/DnrJ/EryC1/StrS aminotransferase, cytosinine-like synthase; GouG
atgactgcaaggaacctgacgacccgagcgggcgtcatcaaccctcaccagctcttcgacctttctcaggaggacatcgactccttcagccacctgaagagcgtgctcgccgacggacagctcttccgctacgtcgagagcgaccgggaatcggcgaacaccttggtcgaacggcacttcgccgagcacttccgcaaggagagggcagtggccgtggccaacggcaccgtaggcctgcgcctggccctgcgtgcgctgggcatcggccccggtcaccgagtggcggtcaatgcctacgctttcatcgcatgtgccatggcgatatcagcgaccggcgccgagccggtgccggtcgacatgggcggatccgtcctgagcatggacgccgacgctctggagaagggcgtgggccacctcgatgcggtcctcctggtgcacgtccagggccacgccgtcgcggccggtccgatacgtgccgtctgcgaccggctcggcataccgatgatcgaggacgtgtgccaggcgctgggagctggttcgtcagaggcgggcgccggcggcgtaggtgacgtcgctgtgacgagtttccaacaggccaagcagatctcctcgggtgaaggcggactcgtcgccgggcccgaggaggtgatcgaacgggtctaccgcctgtcggatctgggcgccgtgcgccaggagaacggtctgccggactgggaccacgaggatgccctgatcggtgacaacctgcggatgactgagcttcaggccgcccttgtcatggatcaggcagtgcggctggagaacacgcttgcccggcaacgggaccagcggtcacggctgcgggccgggctcagtgatatccccgttatcgagagcgagaacccggccgaggacaccggatcgcacacgcttgtcctggcccgggacaccacggcggcggaggagttccgcgttgagctcgcacgccgcggggtgctggcccgaccggtctggaagaagagctgggtggaatacggtttgtaccgacgggagttcgcgagcggcgcccctgccggcccgtggcccgggaaggctgtcggcctcgcctcgcggattctgagtattcccacttcgaaatatgtgacggactccgccgtcgcccaagtggcccaggccatcgcggcgggccgccaccacctcacacaggacaggtga

SEQ ID NO: 16 - protein, 397 aa, Streptomyces microflavus
  MISC_FEATURE (1)..(397): orf4258; DegT/DnrJ/EryC1/StrS aminotransferase, cytosinine-like synthase; GouG
MTARNLTTRAGVINPHQLFDLSQEDIDSFSHLKSVLADGQLFRYVESDRESANTLVERHFAEHFRKERAVAVANGTVGLRLALRALGIGPGHRVAVNAYAFIACAMAISATGAEPVPVDMGGSVLSMDADALEKGVGHLDAVLLVHVQGHAVAAGPIRAVCDRLGIPMIEDVCQALGAGSSEAGAGGVGDVAVTSFQQAKQISSGEGGLVAGPEEVIERVYRLSDLGAVRQENGLPDWDHEDALIGDNLRMTELQAALVMDQAVRLENTLARQRDQRSRLRAGLSDIPVIESENPAEDTGSHTLVLARDTTAAEEFRVELARRGVLARPVWKKSWVEYGLYRREFASGAPAGPWPGKAVGLASRILSIPTSKYVTDSAVAQVAQAIAAGRHHLTQDR

SEQ ID NO: 17 - DNA, 1137 bp, Streptomyces microflavus
  misc_feature (1)..(1137): orf4259; phosphoglycerate mutase; GouH
atgtcatccttcgcgctcctgctccgcggcctgccgaactccggcaagacgaccactgccgcgctgcttcgcaacgccttgaagccgtccgttcggatctccaacgactcggtgcgctacatggcacagccccgggatttcagcgacttcactctcgtcgcctccgagctcggctgcctggatctcgcctcttcatacctggagagcggcttcgtacccgtgatcgacggcgtgttcgaggacatcgacttcctgtccgcgcagaagttgcgcttccacaggaaaggtatgcggctgatcgtcatcaccctggagggaagtctttccgatctgctcgaccgaaacgcctcccgcgatccgctggcccggatggaggaggaccggatgcgagagctccacgcccagttccggccgagcggactcgtcctgtccctcgacgggaaacagcccgaagaggtggcggacgacgtattggacctcctggaattgcagcccccgtaccaggccgaggcagctgacccgggagcggccgacattctcttcctgcgccacggtgctcccgagtaccccagtgacgtctaccccgatccctatgcgatggggctgtccgagcaaggctttgacgaggcccgcgtggcgcgtgccgctgtggagcggttcgcgcccgagatcgtctacacgtccgacttccgtcgtgcggagcaaactgcctcgctggtgaccgccacgatcgatgtcacgccccagcccgaacaccgtctgcgggagcgagtcttccatcagctcgccggcgtagagctcgaagaggtccgctcacagctgggggctgaggcggatgcggtcctcgggggcaacagcgatctgtgcgagcgtgaggaggaggaatcctacgaggctgcaagagcccgggtgctcgcctttttcgatgagatggccgagcggcacgccggccggcgggtcctggtcgtcggccacggcggacctcacgcatggctggtggagcgggcgcttggcgccgagatgcgaggagtgcgccgcatgcgctgggacacgggtcacttctcgcggttcaaggtgacgcccaaccaggtctcactggactacctcaacaggtcaccggaagacgtcaccccatga

SEQ ID NO: 18 - protein, 378 aa, Streptomyces microflavus
  MISC_FEATURE (1)..(378): orf4259; phosphoglycerate mutase; GouH
MSSFALLLRGLPNSGKTTTAALLRNALKPSVRISNDSVRYMAQPRDFSDFTLVASELGCLDLASSYLESGFVPVIDGVFEDIDFLSAQKLRFHRKGMRLIVITLEGSLSDLLDRNASRDPLARMEEDRMRELHAQFRPSGLVLSLDGKQPEEVADDVLDLLELQPPYQAEAADPGAADILFLRHGAPEYPSDVYPDPYAMGLSEQGFDEARVARAAVERFAPEIVYTSDFRRAEQTASLVTATIDVTPQPEHRLRERVFHQLAGVELEEVRSQLGAEADAVLGGNSDLCEREEEESYEAARARVLAFFDEMAERHAGRRVLVVGHGGPHAWLVERALGAEMRGVRRMRWDTGHFSRFKVTPNQVSLDYLNRSPEDVTP

SEQ ID NO: 19 - DNA, 120 bp, Streptomyces microflavus
  misc_feature (1)..(120): orf4260; hypothetical protein
atgctgtacgccttcgaggcaggaccgaagccgaagggggtggccaccacggcgacccgtggccgtcgtcgtggatccgaagcggtcgtggtcgcgtcctttgcgttagtcatggggtga

SEQ ID NO: 20 - protein, 39 aa, Streptomyces microflavus
  MISC_FEATURE (1)..(39): orf4260; hypothetical protein
MLYAFEAGPKPKGVATTATRGRRRGSEAVVVASFALVMG

SEQ ID NO: 21 - DNA, 1116 bp, Streptomyces microflavus
  misc_feature (1)..(1116): orf4261; cytosylglucuronic acid synthase; GouI
gtggccacccccttcggcttcggtcctgcctcgaaggcgtacagcatcggcgaagtcctgcacacccattggggtgtggacgtccagtactacggaacggactccgcccgcgacttcttctccgcgcagcccgatgtgaggcccctggcgccggaggcagtcggtgacaccggagcgatcgacgccgtactgaacgtgctggctccggatctgatccgaagttccgaggaggcagcccggacgtactacgtcgacagcctcggcttcatgtggcagccctcggacattccggacggcagtctgctcaaaagggtgcaccggtacttcgcccaggacatctttggcagcgctgaccatctcactgcgctggggatcaccggagtgacccccgtctcgggaatcgtcgccgaaacagcgccgaccgacatgtcaccgcggccacgctccgtgaaacggctgctcgtccaactcggtggtctgagtaacccggccggccggtcgtccggagaggtttatctcgcactcgccgcgagactactcacggcactgcagcaggacccgtacgaactgagcattgccatgaaccgcgcaggcggcacgttctccctgggatcgctcgaccaggcccgccaattgtccggccgcgacttccacgttgaactggccacctgcgccggtgtcctcagctcacccggtatgaccacgctcattgaggtgtcgcgtgccaagtgcccctatgttccattaccgcctcagaactggagccaggtagtcatatcacgctatatggcgcgaaattcacgcttggggatatgggactttctgatcggtccgtacgccacggtggacgctcatgtccccgaggctcagaaggccgcccaggtgggggagatcaaccagctgctggcaaggaacgccggctacacgacggcctatgtggacctggcccggacggcactggccgaagctcgagtaccggacgtgggcgcaccgttcgacggggcgcgcgtcgtggccgctgccattgcagacgatctcaccaagggaaaatcgcgttacggacggggctcgcatacggatttgaaagaccacggcacaccgactggtgaactctga

SEQ ID NO: 22 - protein, 371 aa, Streptomyces microflavus
  MISC_FEATURE (1)..(371): orf4261; cytosylglucuronic acid synthase; GouI
MATPFGFGPASKAYSIGEVLHTHWGVDVQYYGTDSARDFFSAQPDVRPLAPEAVGDTGAIDAVLNVLAPDLIRSSEEAARTYYVDSLGFMWQPSDIPDGSLLKRVHRYFAQDIFGSADHLTALGITGVTPVSGIVAETAPTDMSPRPRSVKRLLVQLGGLSNPAGRSSGEVYLALAARLLTALQQDPYELSIAMNRAGGTFSLGSLDQARQLSGRDFHVELATCAGVLSSPGMTTLIEVSRAKCPYVPLPPQNWSQVVISRYMARNSRLGIWDFLIGPYATVDAHVPEAQKAAQVGEINQLLARNAGYTTAYVDLARTALAEARVPDVGAPFDGARVVAAAIADDLTKGKSRYGRGSHTDLKDHGTPTGEL

SEQ ID NO: 23 - DNA, 714 bp, Streptomyces microflavus
  misc_feature (1)..(714): orf4262; hypothetical protein; GouJ
atgacgcagcagatcgacaacggcctcgtggccgtacttcagtcgctcgcgcacgaggtggaaaccgcgcgcgagtggagccaggtatcgcggacgctggcacaggagcgggtggccaccgtcttcggctcggcccgtacgcgccgcggtgaaccggcatacaacctggcgtatgaactcgccacggcactggccgcagggaagtggaccacgattaccggcggtggacccggcatcatgcaggccgcgcgggacggcagtggggagggcttgtcccgagcggtgcgggtggagatccccggtgaggaacccgacaccgtgctggacccgtccaggtccataaccgtcgcaaccttcgcgctgcgcaaattactcctgacccacgacatcgacgctctgttcgtcttccccgggggtgtcggcaccttcgacgagctgtacgaggtgctggtccaccaggacaccaaccgacttgcctggttcccggttgtcctgatgcagccggccggcgagagcctctggtcggcctggctggagttcatggagaagcacttggtcagcacgggactggccagctcctccgtgatcaagaggctggttgtggccgagtcggtggaagaggccctggcagccgccgaggggccccgcgcgacggcctacggaacgagcggttctccgtcgcccggaacaggtcacggggcgaccggaaagtga

SEQ ID NO: 24 - protein, 237 aa, Streptomyces microflavus
  MISC_FEATURE (1)..(237): orf4262; hypothetical protein; GouJ
MTQQIDNGLVAVLQSLAHEVETAREWSQVSRTLAQERVATVFGSARTRRGEPAYNLAYELATALAAGKWTTITGGGPGIMQAARDGSGEGLSRAVRVEIPGEEPDTVLDPSRSITVATFALRKLLLTHDIDALFVFPGGVGTFDELYEVLVHQDTNRLAWFPVVLMQPAGESLWSAWLEFMEKHLVSTGLASSSVIKRLVVAESVEEALAAAEGPRATAYGTSGSPSPGTGHGATGK

SEQ ID NO: 25 - DNA, 636 bp, Streptomyces microflavus
  misc_feature (1)..(636): orf4263; glycosyltransferase; GouK
gtggtgacgctcgacgccgaccaagtggtggagccgctcttccttgccgaacacgcccggctgcacgcaggcggccccggccgcgtcgttgcaggccagcgtctccaactcgccgaggggccaatgaacgaggctcgcttggagcacgggttcgacccgcaggccctcccaccggtggtccggggcgacgagcgggagcagctgtttcgtctcctcgacagctccctagaggacatggtgaccggctggcaccacgtctggacctgcaacgcctccttcccccgggacaggctcgaggcggtcgggggcttcgacgagacgttcaccggctgggggctggaggacgcggaactcgcctatcggctggttcaaggtggtgcaaccacgcacttcgccccgtcggcggtggtccgccacgagcaccgcacaccggttacagccgacatgtaccgggagtggtgccgcaacttggcctacttcgtgcgccgacatcccgcaccagaggtacggctccaggagatattcgctcccgccatcgatcccgaccggtccgcgccgggaacgtgggacgacatcgccgccgagttcgagcacacggctcgacggctcggcgctgaccccggccagcatccgtag

SEQ ID NO: 26 - protein, 211 aa, Streptomyces microflavus
  MISC_FEATURE (1)..(211): orf4263; glycosyltransferase; GouK
MVTLDADQVVEPLFLAEHARLHAGGPGRVVAGQRLQLAEGPMNEARLEHGFDPQALPPVVRGDEREQLFRLLDSSLEDMVTGWHHVWTCNASFPRDRLEAVGGFDETFTGWGLEDAELAYRLVQGGATTHFAPSAVVRHEHRTPVTADMYREWCRNLAYFVRRHPAPEVRLQEIFAPAIDPDRSAPGTWDDIAAEFEHTARRLGADPGQHP

SEQ ID NO: 27 - DNA, 1341 bp, Streptomyces microflavus
  misc_feature (1)..(1341): orf4264; hypothetical protein; GouL
atgcccacccctgcgggaaacgtccccgaccatctcgccccgaccgtccgccgggtcgtctatctgcctgtcaaccgccccttcgagacagcatttcactccgtggccgccgaagtggcatcgttggagaagaaccagcgagacaacgtcaccctcctcgtcgtggacgactgtgcgccaccagtgtcacgggccaatcgccaggtgaccgagcgggtagcccgtgaatcgggcctgcgcgtacacacactggaccaacaagactggcttcgtctggccaccacagtgatcgcagcttccgggctgaccggggccgaccgagccacggcgcggaccgccctggtcaaacccaccggttcctacggggcaggtcccaacaaggccgccctggtcgccgcgctggaaggtgcagtctccctgcatcgccgggacagcgaccaggtcacgacggtagaccccgacaccggagcctccccgctccgcctggaagccgacctcctgggtcgcgcccgcccggagggcggcgccgcggcctactgcgcaggctccttcctcaccggtcgccccacgcgagaccgaagggacctggaacgcgactcgacggagtacgcggcccgtatcgacgcactgagccaactcccctccgccccggcccgacgcccgcctctcccgcctgtccgggaacggacggaactcctaggagggcagcacgccgagcgtgacctgacaggtgtggtcgagatggggatcgcggccatgcgaagcgtgtacgagtggatcccagaaatgcccgccgtgggcatcctcggcagcgactacttccagaagggactgctctatcagctcaacctcccggtcttccaccacagcctcccagcccggcacacctacgaatcctggcgcacggagcagtgcgacgattcccatctggcctggtacgtccgggcggaggtgcgctacgccgtactgcgccgccactggaacagcttcaaccacctgctcgtgggccaacggacgcgcgtgctgtccgatggtcacttcgactcccgggcttacggtcagctgttcgtcgaagcgctccacgagggcacccggggggcggaaagcattcccgacgacttcgcggccgtataccgcgacgccgcgaacgcggccacaggcgaggtccgtcgacgtcttctggtgcggctggccgcactggaagaggagaccgatgctgtcaacgcatacgtggccgatgccatccacgaattcgccgccctctcacgtctgtggcccggattgatctctgccgcacaacgggtcgggaggacaaccgcgctggagacgtttacccactga

SEQ ID NO: 28 - protein, 446 aa, Streptomyces microflavus
  MISC_FEATURE (1)..(446): orf4264; hypothetical protein; GouL
MPTPAGNVPDHLAPTVRRVVYLPVNRPFETAFHSVAAEVASLEKNQRDNVTLLVVDDCAPPVSRANRQVTERVARESGLRVHTLDQQDWLRLATTVIAASGLTGADRATARTALVKPTGSYGAGPNKAALVAALEGAVSLHRRDSDQVTTVDPDTGASPLRLEADLLGRARPEGGAAAYCAGSFLTGRPTRDRRDLERDSTEYAARIDALSQLPSAPARRPPLPPVRERTELLGGQHAERDLTGVVEMGIAAMRSVYEWIPEMPAVGILGSDYFQKGLLYQLNLPVFHHSLPARHTYESWRTEQCDDSHLAWYVRAEVRYAVLRRHWNSFNHLLVGQRTRVLSDGHFDSRAYGQLFVEALHEGTRGAESIPDDFAAVYRDAANAATGEVRRRLLVRLAALEEETDAVNAYVADAIHEFAALSRLWPGLISAAQRVGRTTALETFTH

SEQ ID NO: 29 - DNA, 1764 bp, Streptomyces microflavus
  misc_feature (1)..(1764): orf4265; similarity to asparagine synthase; GouM
atgtgcggcatcggaggcatcgtcctgaagcagcgttcccgggtcgacagaaaccacatgatggaacgcctacgcgccggtctcgcccaccgcgggcgaagctcgcagggagacttcgccgacacccgcgcagccttgcactgcgcacggcacgctgtcatcgccgtcgaagccgcccaacagccgctacgggactcccggaacgaactggtgatggtcggcaacggcgagatcctgaactaccaggaactggcacagcagcttcccgccgcgcgaacacgccgcctgctgccaggagacctccaggtcgcgctcgaaatgtacgccgagcacgggatcggcgccctggagaagctgcgcggcccgttcgcggtcgccttgtgggacagccagtcgggcgagctgaccctcgcgcgtgaccggttaggagagcgccccctctacttcttcgactgtcccgactttttcgccttcgcatcggaggtacggtgcctcgccgcagctcttccgcaaggcctgctgaacctggacgaagaagcggcggtagccttcctggggctgggacgagtgcctacgggccgaaccctctaccgggagattttcgcggtcccggcgggctcagtgctccgaatggggactcagccgttcgagccccggcacatggcgtccctggctcctgtctcggcactgcgcagcgcgccggccccgcaggaggagatcgacgccgttctcgcccaggcccaacgccgggcgctggtggcggaccaccccgtagctgtgggattctccggcggcatggattctgcagcagtcctgtcagcggccctggacggagcgggtgccgccgcagtcatcacggtctactccgaactgtcgccagtcaccgatgtgaacctccggcgcgcgcggagtcttgcgccccttctgggcgtccaactgaccgaggtcccgttccgaatgcccacggtccagggcgccgtggatattctgaactcgacactcgacggcccagccgctgagcccctggtgctgcacaacgacgccctgcatactgcggcccgggagcactccgccgtcgtggtcggagggcacggagccgatgaagtcttcggagggtacgcccggtatccggcgctacgagcccaggagaccgcgccgacgaagcactggctcgcgtcgtccgcctgggagcggtggaatcgatcggccgcctggcaccggttcgtcgaggaaaacgccgcgccgcacctggcggatcgcgtatcggcgtcctttcccgatccgctagaaaagaccttcccctacgcatggtcggaatcgagcgaccccgtgctcttcggccaggctctcgatatgttccggctgatgacctacgacaacttccgagctacggacgagaacggcatggcacgacaggtggaggtcagatctcccttcttcgacctcgacctgctggctgccgtctacagtctcccggtgactgagcgcctcggaccaggggcctccaagccgctgttgcggcgttcgttccgaaataccccgctggcaggagcattcaccgaaccgaaggtaggcttcgacgatcatttctcctatgccgactggatgacagagaactggctccagttctcaaccgccatcaccgacgggccactcaaagggacgggcatcctgcaggacggcgccctggacgatctggagcatcgcgactggcgcacactgtggcgccttttcagtctgtccgcctggctgaggcgcagttga

SEQ ID NO: 30 - protein, 587 aa, Streptomyces microflavus
  MISC_FEATURE (1)..(587): orf4265; similarity to asparagine synthase; GouM
MCGIGGIVLKQRSRVDRNHMMERLRAGLAHRGRSSQGDFADTRAALHCARHAVIAVEAAQQPLRDSRNELVMVGNGEILNYQELAQQLPAARTRRLLPGDLQVALEMYAEHGIGALEKLRGPFAVALWDSQSGELTLARDRLGERPLYFFDCPDFFAFASEVRCLAAALPQGLLNLDEEAAVAFLGLGRVPTGRTLYREIFAVPAGSVLRMGTQPFEPRHMASLAPVSALRSAPAPQEEIDAVLAQAQRRALVADHPVAVGFSGGMDSAAVLSAALDGAGAAAVITVYSELSPVTDVNLRRARSLAPLLGVQLTEVPFRMPTVQGAVDILNSTLDGPAAEPLVLHNDALHTAAREHSAVVVGGHGADEVFGGYARYPALRAQETAPTKHWLASSAWGRWNRSAAWHRFVEENAAPHLADRVSASFPDPLEKTFPYAWSESSDPVLFGQALDMFRLMTYDNFRATDENGMARQVEVRSPFFDLDLLAAVYSLPVTERLGPGASKPLLRRSFRNTPLAGAFTEPKVGFDDHFSYADWMTENWLQFSTAITDGPLKGTGILQDGALDDLEHRDWRTLWRLFSLSAWLRRS

SEQ ID NO: 31 - DNA, 114 bp, Streptomyces microflavus
  misc_feature (1)..(114): orf4266; hypothetical protein
gtggacgaggtggaggaagacattggcggtgatgtcgacgtcgtcggccgtgctcggcccaccctcgctaccaagggctccggcgagaactacacggtcaacgacaactcgtag

SEQ ID NO: 32 - protein, 37 aa, Streptomyces microflavus
  MISC_FEATURE (1)..(37): orf4266; hypothetical protein
MDEVEEDIGGDVDVVGRARPTLATKGSGENYTVNDNS

SEQ ID NO: 33 - DNA, 405 bp, Streptomyces microflavus
  misc_feature (1)..(405): orf4267; hypothetical protein
atgtacgagggcggtcttttgcctctgggagcaaagtccgacagctgggtcagttgggaagctgactgttggctgagtcctcagccgttcgacggcaccgccctacgcaagctcgaccggcctttcccgcttgtagaggaatggcagtgggagtacgagtactacgaccacgccctccactcagcgccgctgcacgagatctaccagcacggctccgtgctgctgggcagtgatcaacctggcgactactggacgttggtggtgactggcccgcagcgcggcaaggtgtggtggctcagagacggatgcgccacaccctattcttcgtccggagaacttggagtcggcttcctggactgggtgagggactggcacctcggacagggctgttggcgctccgagtag

SEQ ID NO: 34 - protein, 134 aa, Streptomyces microflavus
  MISC_FEATURE (1)..(134): orf4267; hypothetical protein
MYEGGLLPLGAKSDSWVSWEADCWLSPQPFDGTALRKLDRPFPLVEEWQWEYEYYDHALHSAPLHEIYQHGSVLLGSDQPGDYWTLVVTGPQRGKVWWLRDGCATPYSSSGELGVGFLDWVRDWHLGQGCWRSE

SEQ ID NO: 35 - DNA, 1281 bp, Streptomyces microflavus
  misc_feature (1)..(1281): orf4268; hypothetical protein;+
atgtccgaccaaggaacacacaagctcagcacgaaggccgtgaggatgagtctgtcgcatcacgtcgcccggggggatccgtttgcgggactgtcacgcttccgggatgagttctattcctgtctgaccaggcgtgcggacgcgctgttcgaactcgcggacgcggtgttgtgcgcggacggtccggtccggtcgctggtggaactgtcgctggtcggtgaacaccgtcgcgggcacggcgggctctacgatgccctgtccgcaggccgggtcgatgtcgcccgactgcggcgggccctggccatggtgcggctgcctcgggcggccgacggacggctggtcctggccgccgatctcacctgctggctgcgacccgacgcgcacacctcaccgcagcggatcctgtgccacacctacgggcggggcaaggaccagcacattcccgttcccggctggccctactcggtgatctgcgcacttgagacgggccgtaattcctggaccgcgccgctggacgcactacgtctggcgccgggcgacgacgccgccaccgtcaccgccaggcaggcacgcgagctcgtcgagcggctgatcgatgccgggcagtggacggacggcgatcccgagatcctgatcgtcgtggacgccggctacgacgttccccgcctggccttcctcctgaaggatcttccggtgcaggtgctgggccgaatgcgctcggaccgcgtcctgcgacgcggggtcccaccccgcgagcccggtgtccggggccgcccaccacgccacggcggggagttcgtcttcggtgacccggccacctggaacactcccgacgcacagacggtgaccgcgacacgtctctacggcaccgccgtcgcacgggcatgggaccggctccacccgagactgacccaccgctcggcctggaccgcccagctagtgccgcatcaagcaacgtttgccctgtcaggccgacatactgtgggcgtgcttgagtacttgcttgcgaccgatgacgacgatttcctcgggccggtgcgctcggataggtgtccggatgtgcttcagccaaggggctggggggcggtgccagtggctggcgatggccactatcggatcgctgtcgaaggcatggagattgagttcgcttgggaaatgcccggccttcaggtcacggttcacggcatatcagacgagcccagggttgaggagctggtggctacgatcgcgcgtcaggtcggaaccgagctcgcggtgcgagtccgggtaatcccgctctag

SEQ ID NO: 36 - protein, 426 aa, Streptomyces microflavus
  MISC_FEATURE (1)..(426): orf4268; hypothetical protein
MSDQGTHKLSTKAVRMSLSHHVARGDPFAGLSRFRDEFYSCLTRRADALFELADAVLCADGPVRSLVELSLVGEHRRGHGGLYDALSAGRVDVARLRRALAMVRLPRAADGRLVLAADLTCWLRPDAHTSPQRILCHTYGRGKDQHIPVPGWPYSVICALETGRNSWTAPLDALRLAPGDDAATVTARQARELVERLIDAGQWTDGDPEILIVVDAGYDVPRLAFLLKDLPVQVLGRMRSDRVLRRGVPPREPGVRGRPPRHGGEFVFGDPATWNTPDAQTVTATRLYGTAVARAWDRLHPRLTHRSAWTAQLVPHQATFALSGRHTVGVLEYLLATDDDDFLGPVRSDRCPDVLQPRGWGAVPVAGDGHYRIAVEGMEIEFAWEMPGLQVTVHGISDEPRVEELVATIARQVGTELAVRVRVIPL

SEQ ID NO: 37 - DNA, 1137 bp, Streptomyces microflavus
  misc_feature (1)..(1137): ORF4269; Mobile element; -
gtggctgaacgagtacgcgtccgtgagatcgatgacgatgaaggccggcggttgttgcggatcgtccgccgcggcacagggtcagtggtgacctggcgccgggcccagatggtgctgctgtccgcgcagagcatgcccgtggtgaagatcgccgaggtcgcgttcaccagtgcggaccgggtccgggacgtgatccacaatttcaaatccgacgggttcgcatcgctctacccgaagtacagaggcggccggccggagacgttcaccctgcccgagcgccgggagatcaagaagatcgccaagtccaagccagtcgagcacaatctgcccttctcgacctggagtctggtcaagctggccgacttcctagttgccgagggggtggtcgacgacatcagccacgagggcctgcgcatcctgctccgcgaggaaggcgtctcctttcaacgcgtgaaaacctggaagacctcgaaagacccggactacgcgcagaagaaggcccgtgtcgagcatctctacgccatcgccgacggcgaggtcatacccgaggacggcgagcccgagatcatcttctgcatggacgaattcggcccgctcaacctccagccccaccccggacgccagtgggccgaacgcagtggccggcacaagaaccccgaccgagccccccggccacggcggcgggcgacctacacccgcccgcatggggtccggcacctgttcgccgcctacgacctgggcaaagaccagctctatggtcacatcaagaagaccaagaaccgctccaagtacctggagttctgccgctacctgcgctccttgcacccagcgaaggtgcggatcgccatcatctgcgacaactactccccgcacctgacgacgaagcggtgccaacgggtcgcgacgtgggcggacgcgaacaacgtcgagatcgcctacaccccaaccaacagctcctggctcaaccggatcgaggcccagttcaccgccgccctgcgctacttcaccctcgacggcaccgaccacgccagtcacaaggagcagggcagcatgatccgccgttacatcatctggagaaaccaccacactcatgaccagcaactacgaaccgtagtcaaccaagcccacgttgcctga

SEQ ID NO: 38 - protein, 378 aa, Streptomyces microflavus
  MISC_FEATURE (1)..(378): orf4269; Mobile element protein
MAERVRVREIDDDEGRRLLRIVRRGTGSVVTWRRAQMVLLSAQSMPVVKIAEVAFTSADRVRDVIHNFKSDGFASLYPKYRGGRPETFTLPERREIKKIAKSKPVEHNLPFSTWSLVKLADFLVAEGVVDDISHEGLRILLREEGVSFQRVKTWKTSKDPDYAQKKARVEHLYAIADGEVIPEDGEPEIIFCMDEFGPLNLQPHPGRQWAERSGRHKNPDRAPRPRRRATYTRPHGVRHLFAAYDLGKDQLYGHIKKTKNRSKYLEFCRYLRSLHPAKVRIAIICDNYSPHLTTKRCQRVATWADANNVEIAYTPTNSSWLNRIEAQFTAALRYFTLDGTDHASHKEQGSMIRRYIIWRNHHTHDQQLRTVVNQAHVA

SEQ ID NO: 39 - DNA, 564 bp, Streptomyces microflavus
  misc_feature (1)..(564): orf4270; Transcriptional regulator, MarR family; -
atggggaatccggcacaggagccgtgggagacgtcgcaggtcaagatgatggaagcactgcgggagtgggcgaccggcttcgccgagatcaacctgtacatgtcgcagtggatgcgactgccaggctcggacgccaacgccgtcggacagatcgtctgggcggcacagagcggcacccccctctcccctgccgcactctcccggcgtatcggaatgtccacaggatcaacagccgttctcctgaaccgcctcgaacgggcagggctggtggtccgcagccgcgagcaccaggaccgccgccgcgtcaccctgcggcccacacccgccgcctccgaacaggcacacgcattcatggccatcgccggaaccgagatcgcagcgaccctgcgccaggccaccgaggccgagctcagcaccgcgacctcagttctagatcggatgaacgatgccgcgaagcaggccatccagcgcctgcacaccgtcggcacccgcactccgcccgagaagcgaagctccatccctcagccgctaatgccgcatcagacaacgtttgccctgttgtga

SEQ ID NO: 40 - protein, 188 aa, Streptomyces microflavus
  MISC_FEATURE (1)..(188): orf4270; Transcriptional regulator, MarR family
MGNPAQEPWETSQVKMMEALREWATGFAEINLYMSQWMRLPGSDANAVGQIVWAAQSGTPLSPAALSRRIGMSTGSTAVLLNRLERAGLVVRSREHQDRRRVTLRPTPAASEQAHAFMAIAGTEIAATLRQATEAELSTATSVLDRMNDAAKQAIQRLHTVGTRTPXPEKRSSIPQPLMPHQTTFALL

SEQ ID NO: 41 - DNA, 1107 bp, Streptomyces microflavus
  misc_feature (1)..(1107): orf4271; monooxygenase; +; GouN
gtgatcgagcgcgcaccggagttccgcgatggcgggcagaacatcgacgtgcgcggcgtcgcgcgggaggttcttgtccgtatgggtctgttcgatgcggtcaaggcgcgcaacacgaccgagacgggcgccgtcatcgtggacgggaatggccaggcgattgcgaccctgccagacggcggaggcaccggggcgacggcggagctggagattctgcggggcgatctcgccggtgttctgcgcgatcacctccccgagggggtggagttcgtctacggcgacaccatcgaggacgtgagcgagcatgccgggcatgcccgcctgacgacggcgggcggccgggagctgcggtgcgatctgctggtgatcgccgaaggggtccgctccacgaccagggggcgcgtcttcgcccaagacaccgtcgaggagcgcgagctgggggtgacgatggtgttcggcacgatcccccgcgtgccgggtgacgacgaccgatggcgctggtacaacgcgcctggcgggcggcaggcccatctgcgcccggacccctacggcacgacgcggaccatcctgtcctacagccccggcgacgacctgctgtccatgagccgcaatgaggccttggcccaggtccggtcgcggtaccgcggcgcgggatgggagacatcacgcatccttgacgcgctggagacctcgcaggacgtctacatcgaccagctcgcgcagatccggatgaaaacttggcaccaaggacacgtcgtgatgctgggagacgccgcatggtgcgtgacccccatgggtggcggaggcgcttccctggcgctgaccagcgcgtacgttctggcggcacagctctccgcacattccggcgatctcgctgccgcgctggcggcatacgagcggtggatgcgcccgctcgtgcgggacgcgcagaacatgccgggctggctgacgcgtttcgcctacccccagagccgggcgggactggcgctgcgccacgtcgccgaccgcgtgttcacctccgcccccttccggcccctagctgcaaagctcacccaggtcgccgagactgaacggacgcttcccacgctccgcccgaccaccggataa

SEQ ID NO: 42 - protein, 368 aa, Streptomyces microflavus
  MISC_FEATURE (1)..(368): orf4271; monoxygenase; GouN
MIERAPEFRDGGQNIDVRGVAREVLVRMGLFDAVKARNTTETGAVIVDGNGQAIATLPDGGGTGATAELEILRGDLAGVLRDHLPEGVEFVYGDTIEDVSEHAGHARLTTAGGRELRCDLLVIAEGVRSTTRGRVFAQDTVEERELGVTMVFGTIPRVPGDDDRWRWYNAPGGRQAHLRPDPYGTTRTILSYSPGDDLLSMSRNEALAQVRSRYRGAGWETSRILDALETSQDVYIDQLAQIRMKTWHQGHVVMLGDAAWCVTPMGGGGASLALTSAYVLAAQLSAHSGDLAAALAAYERWMRPLVRDAQNMPGWLTRFAYPQSRAGLALRHVADRVFTSAPFRPLAAKLTQVAETERTLPTLRPTTG

SEQ ID NO: 43 - DNA, 24959 bp, Streptomyces microflavus
  misc_feature (1)..(21933): Genomic DNA sequence of gougerotin gene cluster plus some flanking sequences
atggtccccgcggaccgggacacgctggtccgccgctggtacgtgcgccagatccgtctggacagcgagggcacggtccgcccggaacggatcgagaccgaggccttcgcacggcccaccgacacctacgctcgcgtggagagcagtcggagaccggcatcgtcgcggccggcgtgatcggcttacctccgcgagcacttcccgcgatcttgatggatctcggcgttccggtcgacctccgcttacgcggagagcacacgatgaccatcttcggcctgctcgtgcgcttcggctcacctccgctcgcgcggagagcacgatccctgggtggtctccaccagcccctcggtcggctcacctccgctgacgcggaggtgtaccggacgcgggtgttctggagaaagacgctcaggtgtggtccttgccgaggcggggatggccccgcctgagatcacacctgagggctgtcccgtagtcctggtggatcagcgcgcggcgtcagatgcggggcatcgtaaggcgtaggggcgttcgcgtactggatgatttcgtgcgcggagaatgcggtgaggtgccgtagctgtcgtggtgcgcccgccaggggttacggggcagcccgaagggcttcgagcaggtgttcgtacgcccgcttcggccgcaggtccgtgtcccatggcagggaatcggccgtgcgcgggggatagatcttggtgtcgctggtggagccgtaccggtcggtgaatccccaggtggagaatgatgtgcagttcggctcctccaggcagactcgcagcttgcctgccatctcgacagcctgggtctctcgcccgtcctcctcgtcctcacctatggggacgtccatctccgacacccgcgcctgaaccccgagttccgccagatcctgcacatggctgcggaaggtctccggggcggtgcggtcgccgggtgtgtactcatggttctggaaaccgacgccgtcgatcggtacgccgcgttctttgagtcgtttgatcagatcgtagagggcgtcccatcgctcgccttcctcctcgaccccgtattcgttgaggaacagctctgccttcggatcggcccggtgggcggccctgaaggcctcgtcgatgtactcctcgcccatggcctcgtaccacgggctctgctcggatcgcagaccgaggttcccattggtgtagctcttctcgtcgtcggacatgggctcgttgaccacatcccattctgcgacttttcccttgaagtgccctgcgacggtggcgatgtgccggagcatggtccggcgtacctcctcggggtcgtcgtcctcccgcatccagttgggcagagcctcgtgccagacgagggtgtgcgcgtgaaccttcatgtcgtttgcgcgggcgaaacgtacgagtaggtccgcgtcgcggaagtcgtagacgccgcggcgtgggtggagaaactgtggcttgaacgcgttctccggagtgagcatggagaactggctccccgcgagcgcccggtaacgtgagtcggtgagcagcgggttctcagccagggcggttccgacgtcgaagggtcgttccacatctgcggcccgcgcacgcagcgtgtcgtcggactgcgcctggcgtagctggggggccttgatcacgcggagcgagtcaccgctctctggccgcgcgatcagttgggtcagacgccatccctcgcggcgctcgttttcgcccgcgtccgcctccagaccgaaccagacagcgccctcggcgaacaccccagggtcctgcacggttcctagctgccgcccgtcggcttttacgcggaatgtgtctccgatgctcgacacagcgagcgtcaccgtcccggagcagccgcagtcgaagtccttggaggtggccggctcatcgccctcgccgtcccatatctgcacccttactcgtccgtcggcggtcacgccgacgcggagtgagggccgctcctgcctccactcgtcgtagatgaccggtacgcgtccgtacagccgcagccatgagtcgccgtcatcgtcaccaacaccggacatgcgcgcggtgacggtgaagtcgccatcggatctgagatgcgggccggccagattcagcggggggttgggctggccgccggaggagtcctgctccacgatgtgccgatccgatgcgctgacccgcaggatgccgttctgtgcggtggtgcccggcatctgcgtccagttgtggttccgcagcaggtcttcgtcgcttctgccgccgatgctttcgccctgccccgtacaaccggccgccaccgccatgacgagcagcatggcgcccagtcggcgccacgtctccccactaagatccacggttccccactctcttgttcgagacgaggtcaacgtctcgaagctgggcagtagttcatcacggatgaggtatggctgccttgtggggcacgtcccagccagggactgtcaaatgagcggtgtaactcgggttgttgagggttactggcgtgctgcggagagtcggccgtcgaaggtgatatcgaaggcgttcagaacggttttccagcgcatggtccagcgggcttggcccttgccggtcgggtcgagcgacatgatcgccatgtagacgcacttcagggcagcctgttcgttcgggaagtgcccccgggctttcaccgcccggcggatcctggcgtagacggactcgatcgcgttggtggtgcagacgatgcggcggatctcggtgtcgaagcgcaggaacggggtgaactcctcccacgcgttctcccagagcctgacgatcgccggatacttcttgccccatgcgtcggcgaactccgcgaaccgatcgagggccgcgtcctcggtcgccgcggtgtagacgggcttgaggaccttggcgatcttgtcccagtcctggcgggcggcatagcggaaggagttccnnnnnnnnnnatggaccacgcatgtctgcacgacggtgcggggccagacggtctcgaccgcgtcaggaagccccttcagcccgtcgcagacgagcatgaggacgtcgctcacgccgcggttcttgatctcggtgaggatgtgcatccagtgcttggcgccctcgccgccgtcgccgacccacagcccgagaatgtcccgccggccctcgacggtgaccgccagggcgacgtagatgggccggttggcgaccgcgccgtcgcggatctttacgtggatggcgtcgatgaacaccaccggatagacggcgtccaggggccggttctgccattcggccatgccttcgaagaccttgtcggtgatcgtggagatcgtctggcgcgacacgtcggcgctatagacctcggccaggtgggcctggacctcgccggtgttcaggcccttggccgcgagtgagatgaccatctcgtcgacgccggacaaacgcttctggcgcttcttgacgatcttcggttcgaaggagccctcgcggtcgcggggcacggttatctccacagggcccacgtcggtcagtacggtcttggcgcgggtgccgttgcgggagttgccgccgttcttccccgccggatcgtgcgtgtcatagccgaggtggtcggtgctctcgccctcgagggccgactccaggagtcgtttggtcagctgctggagcagcccgccctcgccggtcagctgcaggccctccgcctgagcctggcccaccaactcgtcgatcagccggtcgtcaacagacttcgacggcaccgtctcagacggctcgacgggctcggccttggtcacgttgtttttggtcatcgatgcatcttccatgatcgggagttacaccgaacgttttacagtcccgcctctggcgctcccgcgacatcgcctcccgcttcggcgacatcaccgtggagaccatgtacagacatccctccagatgggccgagaccggcctcatccacaaacttggccccggcctctacgccgccacagcatggacccaaacaccccttgcgtgacctgcgaaaacgtcaactgaccggccttgggtcaaggcccgaaggctgctccggcgggtctggggaccaccaggagttcaccgcccggcagcctcgtcctggaggcggccgggattgaggacccgcgtccttccagccgtctccacccgctccagcggccgaagcgcggcgggctgctgacgtacgcgcgaccgggcgaaagcggtcacatctccgagatgaccggcccggggtcggctcccgcatcgatcttgcgaagtgtcttgtaggtgaggcgcaggtcagccgggcttgccaggtaaacggtccggtgcgcgatgcggccgatctgcggccccggctcccgagctgggcccgcaagtacttccagggaagcccggctcgctcgtacccgagaatgcctttggccggagtcgccttgtcgggtcggggagcgagggtctgggtgtctcggcagttggtcagagaggtcggcgggcgtggtagatccggcggtggggtgatgcgcccccgccgtagccgtggtcgacctgttccagaatcaacccggcacgcagtagagcggcttcgagatcgagtgcccgcaggtgggtccaatgctccgtgaaccggaggagaacgcttctgtctgttcggaaggttttgtcgatgaggatgtggtctgggtgggaccttgttcgttcccattcctcgacgggctgcctgctcacttcttcgcagggggcggacagtacccgctgccagtcgctgctctcccgcaggtcgaagttgctgctgacatcgatgtggactgtggcacctggcctcatggcgcgtacggcttccgtcagtgctcgctcccggtcctcgacggtccggagtaactggaacgtggagtaggggatcacgacctgatccgcagtccgggtcaggggaagccggcgcatgtcggcacacacagggtgcagtccaggcacgggtggcatacgtgccaagcgccatcgactcaggtctacggcgaccacccgggagcccgggcgagtggccagagggctggccactcgcccggttcccgcccccagcacgacgatattttgctgcgggggtaccaagttgagccagtactccaggtcaagcctctggttgcgcaacctgtgctggttgtgccagtcgtacagcaaggcttccaagtattgttcgcacatctctcctcgcctccttggttatccctggatttagccattctggccggaaggccgctgatttcgaaggtcggggtggtcctggtcgacggatcgactgtatgccacagagtggcgaagatggcgagatgtgttccgcgtaagtgccttatccagccacgtcggaatccatcattgatccgtggcgcgctaacgccgagtttcggccaacccgaacgatgaatgtgtgcgcggggatgatggggaaaggaagagcttggtcatgtgtcatcctgaacaacggtgctgaggggaggtgttcgagatggtggcgtcccatacagcgcggacgaggtcggagatctggaaggggtttccgttcagtactcgtcgcgagattggcacggtcgggatatcaacgttccagagtcagagcacttccgctcttatgcttctgctgctgtccgttctggtggtcgaggtgacgggttcggcggccagtgctcctttggtcatcgctgccggcgcactgcctgcgctgcttctggtgcgctgggttcggagtgtcaaccgtctcctggaccaccgctgggtgatgatcctctcggacacgggcgcttttgcagtcgcgctggctctcttcctgctatcgcaggccgacgtccaagcctggcaaatctactgcggcgtcgccttgttctccggcttcggtgccttctacctgcccgcaatgcgtggctgggtggccgaccagtcgggcgacatcacccaactcacctggctcaactcgatgctggccgtagctacgcaggcgtccgtcgtcgtcggatgggcacttgggggtgttctggccagcaccgtcggggtcaggttcgctctggggctgtgcgccattgcctacgtatgcggcatcgtgctgcagttcgtcgtgtttgtcgtggtcaaccggacgccgctacgtcgggccgctgcgtcggcggctggcctggtggtgcaacggcgttcttcggtgaaggaggtgccggagacggctatccggcaggagggcggtgcgtggcgcaaggtcttctcgccgcgcaggctcgggttgttcactggctcgctgttggcgatggagctcagccacgtgctggctttcagtatgttcgtcccacttgtcactcaagcttccgctgaccgctcctgggtggcgggcctggccaacgcgtgtttcgccctggcggcgatcggcagtggagtgctcgtctccggaggatcgctgggcaactgggtgcgcagccacacaccaggagtcctcatcgccggtttcgctgtgcagacggtgttcggacttaccgcgagtcagaccgtcttagccgttgccctctacacgctggtcggtgtgctcagcggcggggacaccgtgctgcagagcgaggtgcaggatcgctggcgaagtgtcgggtcctcccaggcgttcgccattttcggtgcggtgtccggaccttcccaactggtcggctccttggtcatcgcttttctgctcctgcacttctcgatcaccgctgtctacgtcggaacgattgtgattctgggattcggttcggcgctccttctggtcatggcgagagcgaagtcgatcaatcccgtggaagaagtcgtcccgtaagtttcaccgaaaatttgatgagtctttacggtgcgtcagagatgtggacggatccgatcctcggatattcgcatggaatgcctgaagaagggaaacccgcgatggaaatagccctctacggtctgggagagatgggctcggagatagcgcgctgcctggcgcggggcggtgcgcgggttcacacatacgatccatccgaatcggtggaagtggaggagaagaacctcattcggtgggacagtgtccggcatgccgcagagaacgcctccgtgcacttggtcgtcgcgaagcacctttccgacgtggagtcacttctcttctccgccgatgggataggcccgaatgcttctcaaaagtccctgatcgcggtgcacaccacactcaccccggaggttgtgcgggacctgcacacccggatgcgggacacccacggacacacgctggtggatgccgcgctcagccgccgcggcggtgttgtgcgcgagggttcgctgtccctgttcgtcggggcaggagacgaggccttcgccgtagcccggccggtcttcgaccgctacgccgacaacgtcgtccacgccggagacgtcggtgccggcatgacggtcaagctttgcaacaactggctgctctacgcgaaccggcactccgcactccaggccatccgaacgggccgtcagctcggcgtggatccggctgtgctcacggacgcgctcgcctcttccaccggatccagctgggcactgtcccactactccgacctggacgaagccatcgtcaccgggcggggcgcaccagcggtcatccgggacaggacagcttcggagcttggcatggcgaagcagatggcggcacaggacggtgaggtgcccaccagcctccaggagaccttcgcccttctggacgtgatgtagccgccttcggacgccagccgatcactgcctgcccgtaccggtccgcagcgcgggccccggctccatgccgctcatgcccggaaggccgatatgcacagcgaaccagttacacgcgagtcgtatgtggagaccgtcgccgagcttcccgtcccggtgtggaactcctccgtcgcaacggagttatacacgcggacgctcaccgtggtctgtcgcaggcgacgcgggggcgctgtggcaggggtctgggtctgcccgctggacacgggcatagagggcagaggtgacgcggcacggcggaagtaccggctgctgccatacgcctccccctggatcgatccgaacctccatcccctggagcggcaccgggtcgctctctcgttgctcgaggtggtcctgcagcgcgtgcacctggtcgagttacccatggatccgtgtttcggcgaagtagctgcactcgctgaggtgggtggagacgtggcgtgccgacacacccgcatgctggacatacgtgagggctgtgacccccgagggggttacagccccaccgtccgcaaccatctgcgggctgcggggcgcgctctgcgtgtggaggccgcagatccgtgtgagttcgacttcgctcgcgccgtcgttggccagtccgaggcggctgtggcggaacggagagccgccggactgcgcctccgccgctctgaaggggcggtccgctgtctcgcggcggtggacgccgaaggcgtcctgcgcggacaggcgttcgtcctgcgggaccgtcacaccgcagtgctcatgcaccagtggttcgaccgtgccgggccgcgcgggacaccgactttgctggtcgatagggcggtcatgcagacactgggatcacccggcgtgaactctttcgattttgaaggaagtgtcgttccgggcatcgaccgcttcatggctggcttcggcgctcaggccgtcggttacgggcagttccgctggcaacaggattggcgggaagaccctgccgggaggttcgggtgaatcggaacgaggatcgggcatccaggtctccggaagaggctgctgcgcctccacctttccttcggagggacgtgcgaaaggccggggaccctggcggcccactccaggtcgccccactcggcgagctcaggcctgaacaggaggcttactggcaggcactctacgacctctacggatccctggtgcagcagtcgcccgcgtacgcccgtgcggtcggcgagtcggggcggtacgtccttgtggtgatgggccaccggggggtgttcccgctgcagctcggctcggacgtgtgcacagcgctcggcggcgaccggccgctcctcgcggacggcaccggccccctgcccctggttcctctggtggccgcggccgcagaggccaccgggctccccgtctacctccctctggtggacgcttcgctcgccgaggcaggagttgtggaggcgttcagcgtgtgggagcggccacccaattcactgatcgactggtcgctcgacggcgccgacctatgggatcgcgtaaccgagcgcggtggttcacagtggagcaggaaacagcggctcatcgagcgggacggcctgattctgtccttcggccggtcgggcgaggcggccgccgaggaggtcctgcggatcgatgaccgctcgtggaagtctgcccacaggcagaacatgcgtgcgcgagagggccaggacaagttgtacgccgggctgatcgggtcaggggtgctcacggctacgttcctgcgggacggcgaccgctctgtcgccttccggcttgactcccgcgtcaaaggccgcctgacctgcttgaagtggtcgtatgacgagtcttaccggcgatactcacccggtgtccatttgctcacgcaggggctgcgccaggagtggtgcgggcgcggcatagaggtggtcgatctgcacgggagtcccgactcactgaaggacctgctgtgcaccgaccgggtgagccgcgtggacctctggtacggcgacccgctggccggcgcgcgccgtgcggccgagcggaccggcttcgacaggcggatgagggcggtccgtgacggtgggaaggggttacgccatgccttcgagtgagtccggtgccccaggggtggaacggcccgacagaggtgccacgggcttcgccggtcccgccgggtacgtcaccgtcggtgaagccgtcgcggaactgacccgtcgcatggcccagcccgcggtcgagttcaccgcctccggtaccgccgctctcgaagcggcactggaggtgctgggcatcggacggggcgatgaagtggttgtgcctgacgtgggatgtcactcggtggccgccgccgtcgtgcgacggggagcgatcccggtgttcacgggagtgggggaggccctgacgctcgatccccggggggtttccctggcctgcggcccacgcacccgcgctgtcgttgccgtgcatcagtacggactgccctgtgatgtgcccggcatcatggaggcggtggggccggacatcccggtgatcgaggatgtcgcccagacgtgggggtcggcggtgggcggtgccccggcggggtcgctcgggaccatcgccgtcatttctttcggttcgaccaagccggtggcgctcggcgcgggcggggcgctcttcgggccggcctcactgatcggcggagcggtctcccgcggcgatggagcggaccggcagctgcttcgccctcccagcgccgctcggttccccgcccccctgctcgcccatcttcccaaagccttggaaagggctgaccggctgctggccttgcgtcgggcagcggtggaaacctttctccgtgggccgctggcccaggagttacgtctgcctccgacgccacccggctcctcctccggatggacccgcacccctctgtatccgatcgcccccgcgacctcggtcacggccgaacacatggagcggctggaggcgtgtcacggcccggttcagcgcatgcacgcgacgccgccgtcggcgctgccgatgttccgcggaagcacaacgcgtgtgacaggcggcggccgtcggctcaccgaacccctactcgtgaagatgggatcaccacgatgactgcaaggaacctgacgacccgagcgggcgtcatcaaccctcaccagctcttcgacctttctcaggaggacatcgactccttcagccacctgaagagcgtgctcgccgacggacagctcttccgctacgtcgagagcgaccgggaatcggcgaacaccttggtcgaacggcacttcgccgagcacttccgcaaggagagggcagtggccgtggccaacggcaccgtaggcctgcgcctggccctgcgtgcgctgggcatcggccccggtcaccgagtggcggtcaatgcctacgctttcatcgcatgtgccatggcgatatcagcgaccggcgccgagccggtgccggtcgacatgggcggatccgtcctgagcatggacgccgacgctctggagaagggcgtgggccacctcgatgcggtcctcctggtgcacgtccagggccacgccgtcgcggccggtccgatacgtgccgtctgcgaccggctcggcataccgatgatcgaggacgtgtgccaggcgctgggagctggttcgtcagaggcgggcgccggcggcgtaggtgacgtcgctgtgacgagtttccaacaggccaagcagatctcctcgggtgaaggcggactcgtcgccgggcccgaggaggtgatcgaacgggtctaccgcctgtcggatctgggcgccgtgcgccaggagaacggtctgccggactgggaccacgaggatgccctgatcggtgacaacctgcggatgactgagcttcaggccgcccttgtcatggatcaggcagtgcggctggagaacacgcttgcccggcaacgggaccagcggtcacggctgcgggccgggctcagtgatatccccgttatcgagagcgagaacccggccgaggacaccggatcgcacacgcttgtcctggcccgggacaccacggcggcggaggagttccgcgttgagctcgcacgccgcggggtgctggcccgaccggtctggaagaagagctgggtggaatacggtttgtaccgacgggagttcgcgagcggcgcccctgccggcccgtggcccgggaaggctgtcggcctcgcctcgcggattctgagtattcccacttcgaaatatgtgacggactccgccgtcgcccaagtggcccaggccatcgcggcgggccgccaccacctcacacaggacaggtgagaccccatgtcatccttcgcgctcctgctccgcggcctgccgaactccggcaagacgaccactgccgcgctgcttcgcaacgccttgaagccgtccgttcggatctccaacgactcggtgcgctacatggcacagccccgggatttcagcgacttcactctcgtcgcctccgagctcggctgcctggatctcgcctcttcatacctggagagcggcttcgtacccgtgatcgacggcgtgttcgaggacatcgacttcctgtccgcgcagaagttgcgcttccacaggaaaggtatgcggctgatcgtcatcaccctggagggaagtctttccgatctgctcgaccgaaacgcctcccgcgatccgctggcccggatggaggaggaccggatgcgagagctccacgcccagttccggccgagcggactcgtcctgtccctcgacgggaaacagcccgaagaggtggcggacgacgtattggacctcctggaattgcagcccccgtaccaggccgaggcagctgacccgggagcggccgacattctcttcctgcgccacggtgctcccgagtaccccagtgacgtctaccccgatccctatgcgatggggctgtccgagcaaggctttgacgaggcccgcgtggcgcgtgccgctgtggagcggttcgcgcccgagatcgtctacacgtccgacttccgtcgtgcggagcaaactgcctcgctggtgaccgccacgatcgatgtcacgccccagcccgaacaccgtctgcgggagcgagtcttccatcagctcgccggcgtagagctcgaagaggtccgctcacagctgggggctgaggcggatgcggtcctcgggggcaacagcgatctgtgcgagcgtgaggaggaggaatcctacgaggctgcaagagcccgggtgctcgcctttttcgatgagatggccgagcggcacgccggccggcgggtcctggtcgtcggccacggcggacctcacgcatggctggtggagcgggcgcttggcgccgagatgcgaggagtgcgccgcatgcgctgggacacgggtcacttctcgcggttcaaggtgacgcccaaccaggtctcactggactacctcaacaggtcaccggaagacgtcaccccatgactaacgcaaaggacgcgaccacgaccgcttcggatccacgacgacggccacgggtcgccgtggtggccacccccttcggcttcggtcctgcctcgaaggcgtacagcatcggcgaagtcctgcacacccattggggtgtggacgtccagtactacggaacggactccgcccgcgacttcttctccgcgcagcccgatgtgaggcccctggcgccggaggcagtcggtgacaccggagcgatcgacgccgtactgaacgtgctggctccggatctgatccgaagttccgaggaggcagcccggacgtactacgtcgacagcctcggcttcatgtggcagccctcggacattccggacggcagtctgctcaaaagggtgcaccggtacttcgcccaggacatctttggcagcgctgaccatctcactgcgctggggatcaccggagtgacccccgtctcgggaatcgtcgccgaaacagcgccgaccgacatgtcaccgcggccacgctccgtgaaacggctgctcgtccaactcggtggtctgagtaacccggccggccggtcgtccggagaggtttatctcgcactcgccgcgagactactcacggcactgcagcaggacccgtacgaactgagcattgccatgaaccgcgcaggcggcacgttctccctgggatcgctcgaccaggcccgccaattgtccggccgcgacttccacgttgaactggccacctgcgccggtgtcctcagctcacccggtatgaccacgctcattgaggtgtcgcgtgccaagtgcccctatgttccattaccgcctcagaactggagccaggtagtcatatcacgctatatggcgcgaaattcacgcttggggatatgggactttctgatcggtccgtacgccacggtggacgctcatgtccccgaggctcagaaggccgcccaggtgggggagatcaaccagctgctggcaaggaacgccggctacacgacggcctatgtggacctggcccggacggcactggccgaagctcgagtaccggacgtgggcgcaccgttcgacggggcgcgcgtcgtggccgctgccattgcagacgatctcaccaagggaaaatcgcgttacggacggggctcgcatacggatttgaaagaccacggcacaccgactggtgaactctgaagaaaagagattccgatgacgcagcagatcgacaacggcctcgtggccgtacttcagtcgctcgcgcacgaggtggaaaccgcgcgcgagtggagccaggtatcgcggacgctggcacaggagcgggtggccaccgtcttcggctcggcccgtacgcgccgcggtgaaccggcatacaacctggcgtatgaactcgccacggcactggccgcagggaagtggaccacgattaccggcggtggacccggcatcatgcaggccgcgcgggacggcagtggggagggcttgtcccgagcggtgcgggtggagatccccggtgaggaacccgacaccgtgctggacccgtccaggtccataaccgtcgcaaccttcgcgctgcgcaaattactcctgacccacgacatcgacgctctgttcgtcttccccgggggtgtcggcaccttcgacgagctgtacgaggtgctggtccaccaggacaccaaccgacttgcctggttcccggttgtcctgatgcagccggccggcgagagcctctggtcggcctggctggagttcatggagaagcacttggtcagcacgggactggccagctcctccgtgatcaagaggctggttgtggccgagtcggtggaagaggccctggcagccgccgaggggccccgcgcgacggcctacggaacgagcggttctccgtcgcccggaacaggtcacggggcgaccggaaagtgaccgccaccgaggacgaccggccccgcagccaggcagatacaaggccgcctggcaccgctcaggtccacatcagcgtgatcatcccaacgttcaacgcccgcaccactttgcgatgctgtcttctctcgctcctccaccagaggctcggcgctcacggaccggacgcagggtctccgttcacgtacgaggtcatcgtcgttgacgacggttcctccgacggcacaaccgaaatgatcgcccagttcccctcccggctcgacctgcgatacaccttcctgcctcgaacggacagatcgggtcgggcccgggcacggaatgccggcctggctctcgccacgggaggcctcgtggtgacgctcgacgccgaccaagtggtggagccgctcttccttgccgaacacgcccggctgcacgcaggcggccccggccgcgtcgttgcaggccagcgtctccaactcgccgaggggccaatgaacgaggctcgcttggagcacgggttcgacccgcaggccctcccaccggtggtccggggcgacgagcgggagcagctgtttcgtctcctcgacagctccctagaggacatggtgaccggctggcaccacgtctggacctgcaacgcctccttcccccgggacaggctcgaggcggtcgggggcttcgacgagacgttcaccggctgggggctggaggacgcggaactcgcctatcggctggttcaaggtggtgcaaccacgcacttcgccccgtcggcggtggtccgccacgagcaccgcacaccggttacagccgacatgtaccgggagtggtgccgcaacttggcctacttcgtgcgccgacatcccgcaccagaggtacggctccaggagatattcgctcccgccatcgatcccgaccggtccgcgccgggaacgtgggacgacatcgccgccgagttcgagcacacggctcgacggctcggcgctgaccccggccagcatccgtagctggcatcaccggttccgctgctatggcagcacccgctcataggccgcagcagcggacccccctggaccaaattgagaggaggcgattcaccgccatgcccacccctgcgggaaacgtccccgaccatctcgccccgaccgtccgccgggtcgtctatctgcctgtcaaccgccccttcgagacagcatttcactccgtggccgccgaagtggcatcgttggagaagaaccagcgagacaacgtcaccctcctcgtcgtggacgactgtgcgccaccagtgtcacgggccaatcgccaggtgaccgagcgggtagcccgtgaatcgggcctgcgcgtacacacactggaccaacaagactggcttcgtctggccaccacagtgatcgcagcttccgggctgaccggggccgaccgagccacggcgcggaccgccctggtcaaacccaccggttcctacggggcaggtcccaacaaggccgccctggtcgccgcgctggaaggtgcagtctccctgcatcgccgggacagcgaccaggtcacgacggtagaccccgacaccggagcctccccgctccgcctggaagccgacctcctgggtcgcgcccgcccggagggcggcgccgcggcctactgcgcaggctccttcctcaccggtcgccccacgcgagaccgaagggacctggaacgcgactcgacggagtacgcggcccgtatcgacgcactgagccaactcccctccgccccggcccgacgcccgcctctcccgcctgtccgggaacggacggaactcctaggagggcagcacgccgagcgtgacctgacaggtgtggtcgagatggggatcgcggccatgcgaagcgtgtacgagtggatcccagaaatgcccgccgtgggcatcctcggcagcgactacttccagaagggactgctctatcagctcaacctcccggtcttccaccacagcctcccagcccggcacacctacgaatcctggcgcacggagcagtgcgacgattcccatctggcctggtacgtccgggcggaggtgcgctacgccgtactgcgccgccactggaacagcttcaaccacctgctcgtgggccaacggacgcgcgtgctgtccgatggtcacttcgactcccgggcttacggtcagctgttcgtcgaagcgctccacgagggcacccggggggcggaaagcattcccgacgacttcgcggccgtataccgcgacgccgcgaacgcggccacaggcgaggtccgtcgacgtcttctggtgcggctggccgcactggaagaggagaccgatgctgtcaacgcatacgtggccgatgccatccacgaattcgccgccctctcacgtctgtggcccggattgatctctgccgcacaacgggtcgggaggacaaccgcgctggagacgtttacccactgagtagtccggccggtcgaactgccgtcggaacgagggaagacaacacccaaccggaaggaagacgccatgtgcggcatcggaggcatcgtcctgaagcagcgttcccgggtcgacagaaaccacatgatggaacgcctacgcgccggtctcgcccaccgcgggcgaagctcgcagggagacttcgccgacacccgcgcagccttgcactgcgcacggcacgctgtcatcgccgtcgaagccgcccaacagccgctacgggactcccggaacgaactggtgatggtcggcaacggcgagatcctgaactaccaggaactggcacagcagcttcccgccgcgcgaacacgccgcctgctgccaggagacctccaggtcgcgctcgaaatgtacgccgagcacgggatcggcgccctggagaagctgcgcggcccgttcgcggtcgccttgtgggacagccagtcgggcgagctgaccctcgcgcgtgaccggttaggagagcgccccctctacttcttcgactgtcccgactttttcgccttcgcatcggaggtacggtgcctcgccgcagctcttccgcaaggcctgctgaacctggacgaagaagcggcggtagccttcctggggctgggacgagtgcctacgggccgaaccctctaccgggagattttcgcggtcccggcgggctcagtgctccgaatggggactcagccgttcgagccccggcacatggcgtccctggctcctgtctcggcactgcgcagcgcgccggccccgcaggaggagatcgacgccgttctcgcccaggcccaacgccgggcgctggtggcggaccaccccgtagctgtgggattctccggcggcatggattctgcagcagtcctgtcagcggccctggacggagcgggtgccgccgcagtcatcacggtctactccgaactgtcgccagtcaccgatgtgaacctccggcgcgcgcggagtcttgcgccccttctgggcgtccaactgaccgaggtcccgttccgaatgcccacggtccagggcgccgtggatattctgaactcgacactcgacggcccagccgctgagcccctggtgctgcacaacgacgccctgcatactgcggcccgggagcactccgccgtcgtggtcggagggcacggagccgatgaagtcttcggagggtacgcccggtatccggcgctacgagcccaggagaccgcgccgacgaagcactggctcgcgtcgtccgcctgggagcggtggaatcgatcggccgcctggcaccggttcgtcgaggaaaacgccgcgccgcacctggcggatcgcgtatcggcgtcctttcccgatccgctagaaaagaccttcccctacgcatggtcggaatcgagcgaccccgtgctcttcggccaggctctcgatatgttccggctgatgacctacgacaacttccgagctacggacgagaacggcatggcacgacaggtggaggtcagatctcccttcttcgacctcgacctgctggctgccgtctacagtctcccggtgactgagcgcctcggaccaggggcctccaagccgctgttgcggcgttcgttccgaaataccccgctggcaggagcattcaccgaaccgaaggtaggcttcgacgatcatttctcctatgccgactggatgacagagaactggctccagttctcaaccgccatcaccgacgggccactcaaagggacgggcatcctgcaggacggcgccctggacgatctggagcatcgcgactggcgcacactgtggcgccttttcagtctgtccgcctggctgaggcgcagttgaactacggaagttgaactcgcgacgtcgctgccgctcaggtgagtctgatggtcaggccggtctcggcgaggcagccgttctgtgaagtcgctgcggtacgcgctggacgtggtgttagggggtactgaaggcgacgttggctcgcagcgtggacgaggtggaggaagacattggcggtgatgtcgacgtcgtcggccgtgctcggcccaccctcgctaccaagggctccggcgagaactacacggtcaacgacaactcgtaggtcgtctgcggcaacgttcccaccgccaacgccacggtctacatcgtcgacaccgttctgatgcccaaggcgtgaccaccccactccgcccctgacgcgcggtgatgcggcaacgctctggtgagcgcgccacacgccgcctcccgctggagtgcaagctccagcaacgggcgagccacatccccatccctgactcggggccgcgtgcgtttctgaaggagcccgaccgctggcgctttcgccggaggccacacggaaggtctgccgggtgagcggctccggtctgcaggcggtggcgaaggccggggacatcacccaggcctagcttgatctttaaccccctgcactatgcatcaggaccgtatgtcggttgctggtgatatctgtggggcgtggaagatgcgatcttgagtgcgatagccctgctaagtgaagccttcgtggcagagacccgtcgccggccgctgggctgggaggggcttcggtcgtgggaggaccagcaaggtgtcgtgttgccggagccgtatcgcaccttcgtggccgagattgcgaacggaacaaacgagggcccgatgtacgagggcggtcttttgcctctgggagcaaagtccgacagctgggtcagttgggaagctgactgttggctgagtcctcagccgttcgacggcaccgccctacgcaagctcgaccggcctttcccgcttgtagaggaatggcagtgggagtacgagtactacgaccacgccctccactcagcgccgctgcacgagatctaccagcacggctccgtgctgctgggcagtgatcaacctggcgactactggacgttggtggtgactggcccgcagcgcggcaaggtgtggtggctcagagacggatgcgccacaccctattcttcgtccggagaacttggagtcggcttcctggactgggtgagggactggcacctcggacagggctgttggcgctccgagtagcccagttcctcaatcgcgaactcgtaggtcggacggttcgtcgtacttgatcactcggactggtaagtgccttacgagcagccgaccttcggctgagcatgtgatccgaccaaggaacacacaagctcagcacgaaggccgtgaggatgagtctgtcgcatcacgtcgcccggggggatccgtttgcgggactgtcacgcttccgggatgagttctattcctgtctgaccaggcgtgcggacgcgctgttcgaactcgcggacgcggtgttgtgcgcggacggtccggtccggtcgctggtggaactgtcgctggtcggtgaacaccgtcgcgggcacggcgggctctacgatgccctgtccgcaggccgggtcgatgtcgcccgactgcggcgggccctggccatggtgcggctgcctcgggcggccgacggacggctggtcctggccgccgatctcacctgctggctgcgacccgacgcgcacacctcaccgcagcggatcctgtgccacacctacgggcggggcaaggaccagcacattcccgttcccggctggccctactcggtgatctgcgcacttgagacgggccgtaattcctggaccgcgccgctggacgcactacgtctggcgccgggcgacgacgccgccaccgtcaccgccaggcaggcacgcgagctcgtcgagcggctgatcgatgccgggcagtggacggacggcgatcccgagatcctgatcgtcgtggacgccggctacgacgttccccgcctggccttcctcctgaaggatcttccggtgcaggtgctgggccgaatgcgctcggaccgcgtcctgcgacgcggggtcccaccccgcgagcccggtgtccggggccgcccaccacgccacggcggggagttcgtcttcggtgacccggccacctggaacactcccgacgcacagacggtgaccgcgacacgtctctacggcaccgccgtcgcacgggcatgggaccggctccacccgagactgacccaccgctcggcctggaccgcccagctagtgccgcatcaagcaacgtttgccctgtcaggccgacatactgtgggcgtgcttgagtacttgcttgcgaccgatgacgacgatttcctcgggccggtgcgctcggataggtgtccggatgtgcttcagccaaggggctggggggcggtgccagtggctggcgatggccactatcggatcgctgtcgaaggcatggagattgagttcgcttgggaaatgcccggccttcaggtcacggttcacggcatatcagacgagcccagggttgaggagctggtggctacgatcgcgcgtcaggtcggaaccgagctcgcggtgcgagtccgggtaatcccgctctagcgctgcttcaggcaacgtgggcttggttgactacggttcgtagttgctggtcatgagtgtggtggtttctccagatgatgtaacggcggatcatgctgccctgctccttgtgactggcgtggtcggtgccgtcgagggtgaagtagcgcagggcggcggtgaactgggcctcgatccggttgagccaggagctgttggttggggtgtaggcgatctcgacgttgttcgcgtccgcccacgtcgcgacccgttggcaccgcttcgtcgtcaggtgcggggagtagttgtcgcagatgatggcgatccgcaccttcgctgggtgcaaggagcgcaggtagcggcagaactccaggtacttggagcggttcttggtcttcttgatgtgaccatagagctggtctttgcccaggtcgtaggcggcgaacaggtgccggaccccatgcgggcgggtgtaggtcgcccgccgccgtggccggggggctcggtcggggttcttgtgccggccactgcgttcggcccactggcgtccggggtggggctggaggttgagcgggccgaattcgtccatgcagaagatgatctcgggctcgccgtcctcgggtatgacctcgccgtcggcgatggcgtagagatgctcgacacgggccttcttctgcgcgtagtccgggtctttcgaggtcttccaggttttcacgcgttgaaaggagacgccttcctcgcggagcaggatgcgcaggccctcgtggctgatgtcgtcgaccaccccctcggcaactaggaagtcggccagcttgaccagactccaggtcgagaagggcagattgtgctcgactggcttggacttggcgatcttcttgatctcccggcgctcgggcagggtgaacgtctccggccggccgcctctgtacttcgggtagagcgatgcgaacccgtcggatttgaaattgtggatcacgtcccggacccggtccgcactggtgaacgcgacctcggcgatcttcaccacgggcatgctctgcgcggacagcagcaccatctgggcccggcgccaggtcaccactgaccctgtgccgcggcggacgatccgcaacaaccgccggccttcatcgtcatcgatctcacggacgcgtactcgttcagccacccgcacagcttgacggccgcacgccgcccaggacaggtcccaaacggcgcgtcaggtcacaacagggcaaacgttgtctgatgcggcattagcggctgagggatggagcttcgcttctcgggctacggagtgcgggtgccgacggtgtgcaggcgctggatggcctgcttcgcggcatcgttcatccgatctagaactgaggtcgcggtgctgagctcggcctcggtggcctggcgcagggtcgctgcgatctcggttccggcgatggccatgaatgcgtgtgcctgttcggaggcggcgggtgtgggccgcagggtgacgcggcggcggtcctggtgctcgcggctgcggaccaccagccctgcccgttcgaggcggttcaggagaacggctgttgatcctgtggacattccgatacgccgggagagtgcggcaggggagaggggggtgccgctctgtgccgcccagacgatctgtccgacggcgttggcgtccgagcctggcagtcgcatccactgcgacatgtacaggttgatctcggcgaagccggtcgcccactcccgcagtgcttccatcatcttgacctgcgacgtctcccacggctcctgtgccggattccccatgcccactccaatgctctactgtagagatactttactgtagagcttagtggacgggagctgtcatgagtgcaggcactgcggtggtgtccggggcgagcatcgctgggctgtcggcggctttctggctgcggcgccttgggtggcaggtcactgtgatcgagcgcgcaccggagttccgcgatggcgggcagaacatcgacgtgcgcggcgtcgcgcgggaggttcttgtccgtatgggtctgttcgatgcggtcaaggcgcgcaacacgaccgagacgggcgccgtcatcgtggacgggaatggccaggcgattgcgaccctgccagacggcggaggcaccggggcgacggcggagctggagattctgcggggcgatctcgccggtgttctgcgcgatcacctccccgagggggtggagttcgtctacggcgacaccatcgaggacgtgagcgagcatgccgggcatgcccgcctgacgacggcgggcggccgggagctgcggtgcgatctgctggtgatcgccgaaggggtccgctccacgaccagggggcgcgtcttcgcccaagacaccgtcgaggagcgcgagctgggggtgacgatggtgttcggcacgatcccccgcgtgccgggtgacgacgaccgatggcgctggtacaacgcgcctggcgggcggcaggcccatctgcgcccggacccctacggcacgacgcggaccatcctgtcctacagccccggcgacgacctgctgtccatgagccgcaatgaggccttggcccaggtccggtcgcggtaccgcggcgcgggatgggagacatcacgcatccttgacgcgctggagacctcgcaggacgtctacatcgaccagctcgcgcagatccggatgaaaacttggcaccaaggacacgtcgtgatgctgggagacgccgcatggtgcgtgacccccatgggtggcggaggcgcttccctggcgctgaccagcgcgtacgttctggcggcacagctctccgcacattccggcgatctcgctgccgcgctggcggcatacgagcggtggatgcgcccgctcgtgcgggacgcgcagaacatgccgggctggctgacgcgtttcgcctacccccagagccgggcgggactggcgctgcgccacgtcgccgaccgcgtgttcacctccgcccccttccggcccctagctgcaaagctcacccaggtcgccgagactgaacggacgcttcccacgctccgcccgaccaccggataa